(19) United States
(12) Patent Application Publication
(10) Pub. No.: US 2025/0345332 A1
(43) Pub. Date: Nov. 13, 2025

(54) APROCITENTAN FOR THE TREATMENT OF HYPERTENSION

(71) Applicant: IDORSIA PHARMACEUTICALS LTD, Allschwil (CH)

(72) Inventor: Parisa DANAIETASH, Allschwil (CH)

(21) Appl. No.: 18/868,080

(22) PCT Filed: May 20, 2023
(86) PCT No.: PCT/EP2023/063538

§ 371 Date: Nov. 21, 2024

Related U.S. Application Data
(60) Provisional application No. 63/344,618, filed on May 22, 2022.

(51) Int. Cl. A61K 31/513; A61K 31/401; A61K 31/403; A61K 31/41; A61K 31/4178; A61K 31/4184; A61P 9/12

(52) U.S. Cl. CPC A61K 31/513; A61K 31/401; A61K 31/403; A61K 31/41; A61K 31/4178; A61K 31/4184; A61P 9/12

(57) ABSTRACT

The present invention concerns the compound aprocitentan, {5-(4-bromo-phenyl)-6-[2-(5-bromo-pyrimidin-2-yloxy)-ethoxy]-pyrimidin-4-yl}-sulfamide: Formula (I) and its use as endothelin receptor antagonist in a method of treating hypertension including resistant hypertension in a subject in need thereof, said method comprising administering to the subject a pharmaceutical composition comprising a clinically proven effective amount of aprocitentan, or a pharmaceutically acceptable salt thereof.

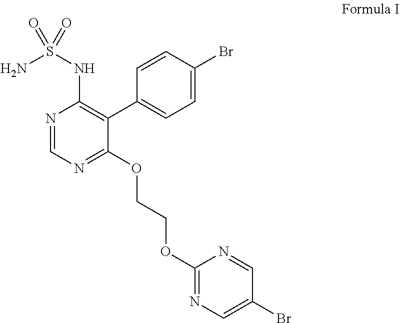 [SMILES: NS(=O)(=O)Nc1ncnc(OCCOc2ncc(Br)cn2)c1-c1ccc(Br)cc1]

The present invention concerns the compound aprocitentan and its use as endothelin receptor antagonist of proven clinical efficacy for the treatment of hypertension including difficult to control hypertension and resistant hypertension.

Aprocitentan, {5-(4-bromo-phenyl)-6-[2-(5-bromo-pyrimidin-2-yloxy)-ethoxy]-pyrimidin-4-yl}-sulfamide (hereinafter also referred to as “COMPOUND”), has the formula I

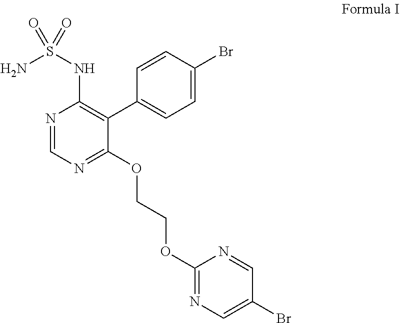 [SMILES: NS(=O)(=O)Nc1ncnc(OCCOc2ncc(Br)cn2)c1-c1ccc(Br)cc1]

The compound of formula I, also known under the name, and referred to as ACT-132577, is an endothelin receptor antagonist. The compound of formula l is a member of a structural family that was previously generically disclosed in WO 02/053557. In particular, the compound of formula I, while showing endothelin receptor antagonist activity, exhibits in vivo a much longer half-life and a much shorter clearance in comparison to corresponding alkylated derivatives. This makes the compound of formula I particularly suitable for long-acting pharmaceutical compositions, as disclosed in WO 2009/024906.

Because of its ability to inhibit the endothelin binding, the compound of formula I can be used for treatment of endothelin related diseases which are associated with an increase in vasoconstriction, proliferation or inflammation due to endothelin occurring in many cardio-renal-metabolic diseases. Examples of such endothelin related diseases are hypertension including especially difficult to control hypertension and resistant hypertension. Further examples of endothelin related diseases disclosed for example in WO 2009/024906, WO2018/153513, or WO2018/154101 are pulmonary hypertension; coronary diseases; cardiac insufficiency; renal and myocardial ischemia; chronic kidney disease (CKD) [especially CKD of stages 1 to 4 as defined by the Kidney Disease Improving Global Outcomes (KDIGO) Guidelines (and notably CKD of stage 3 or 4), and in particular CKD (notably of these stages) caused by/associated with hypertension or diabetes (diabetic kidney disease (DKD), including DKD that is associated, in addition, with hypertension); diabetes, and diabetes related diseases such as diabetic arteriopathy, diabetic nephropathy, diabetic retinopathy, or diabetic vasculopathy; reducing the risk of developing a major cardiovascular event (such as heart failure (HF), myocardial infarction, stroke, or death from cardiovascular causes) in patients who have diabetes, especially in patients who have diabetes that is accompanied by at least one other cardiovascular risk factor (such as hypertension, dyslipedemia, thrombotic phenomenom); therapy and prophylaxis of diabetic complications; (acute and chronic) renal failure; glomerulonephritis; connective tissue diseases; atherosclerosis; peripheral arterial disease including chronic peripheral (obliterant) arteriopathy; digital ulcers; diabetic foot ulcers and/or reducing the risk of lower limb/extremety amputations in patients who have diabetes, or who are smokers, or who have atherosclerosis; heart failure (HF) defined as including especially chronic HF, including in particular systolic HF/HF with reduced ejection fraction (HFrEF) (i.e. ejection fraction <about 40%), and diastolic HF/HF with preserved ejection fraction (HFpEF) (i.e. ejection fraction >about 50%); reducing the risk of developing a major cardiovascular event (such as heart failure (HF), myocardial infarction, stroke, or death from cardiovascular causes) in patients who are at cardiovascular risk (such as patients who have coronary artery disease and/or patients who have demonstrated clinical signs of congestive heart failure); angina pectoris; and diastolic dysfunction. The compound of formula I can also be used in the treatment or prevention of cerebral ischemia; dementia; migraine; subarachnoidal hemorrhage; Raynaud's syndrome; portal hypertension; restenosis after balloon or stent angioplasty; inflammation; stomach and duodenal ulcer; cancer; melanoma; prostate cancer; prostatic hypertrophy; erectile dysfunction; eclampsia; hearing loss; amaurosis; chronic bronchitis; asthma; pulmonary fibrosis; gram negative septicemia; shock, sickle cell anemia; renal colic; glaucoma; complications of vascular or cardiac surgery or after organ transplantation; complications of cyclosporin treatment or equivalent therapies showing nephrotoxic profile; pain; dyslipidemia; as well as other diseases presently known to be related to endothelin.

According to the 2014 American Society of Hypertension and International Society of Hypertension joint statement [Weber et al., “Clinical Practice Guidelines for the Management of Hypertension in the Community. A Statement by the American Society of Hypertension and the International Society of Hypertension.” J Clin Hypertens (2014), 16(1), 14-26], the 2013 European Society of Hypertension and European Society of Cardiology joint guideline [Mancia et al, J. Hypertens. (2013), 31, 1281-1357], as well as several national guidelines [Denolle et al., J Hum Hypertens. (2016), 30(11), 657-663; McCormack et al., Br J Cardiol (2013), 20 (suppl 1), S1-S16], resistant hypertension (rHT) is defined as uncontrolled blood pressure (BP) (i.e., failure to lower BP to a pre-defined threshold) despite concurrent administration of three antihypertensive therapies of different pharmacological classes at maximal or optimal doses, including a diuretic. Thus, resistant hypertension patients include patients whose blood pressure is controlled with use of more than three medications. That is, patients whose blood pressure is controlled but require four or more medications to do so should be considered resistant to treatment (see e.g. Mancia et al, J. Hypertens. (2013).

Clinical studies have shown that endothelin receptor antagonists (ERAs) may have significant treatment effect in patients suffering from hypertension and/or renal disease, whether associated or not with diabetes. Endothelin 1 (ET-1) is likely to play a role in the pathogenic mechanisms of chronic diabetic arteriopathy, because of its effects on mediating plaque formation, thrombosis, vasoconstriction, and vascular hypertrophy and because it potentiates the action of other systems, in particular the renin angiotensin and sympathetic systems and/or insulin signalings. Thus, an ERA might be beneficial in the treatment of peripheral arterial obliterant disease including diabetic arteriopathy by having acute (peripheral vasodilation) and chronic (vasodilation, improvement in vascular structure and modulation of sympathetic activity, antithrombotic, antiinflamatory) effects. In a clinical network meta-analysis of studies performed in adults with diabetes and CKD (157 studies comprising 43,256 patients), ERAs were ranked as the most effective agents for the prevention of end-stage kidney disease (S. C. Palmer et al., Lancet (2015), 385 (9982): 2047-2056). However, therapeutic benefit needs to be weighted against potential side effects, such as the potential risk of teratogenic activity generally associated with ERAs. In addition and more importantly, both, selective ET_{A}-antagonists and dual antagonists of both the ET_{A} and ET_{B} receptor, may cause fluid retention, a common side effect associated with many previously studied ERAs and sometimes (e.g. if not manageable with diuretics) leading to exaggerated major adverse cardiac events such as heart failure or death. Whereas the risk-benefit balance is in most cases in favor of treatment with an ERA for indications such as pulmonary hypertension (as reflected in the past by successive market approvals e.g. for the ERAs the dual antagonists bosentan and macitentan, and the ET_{A}-selective antagonist ambrisentan), it was stated that ERAs have no role in the management of primary hypertension (Laffin et al. Seminars in Nephrology 2015, 35, 168-175), and side effects such as fluid retention may remain an issue when a potential treatment of difficult to control hypertension and resistant hypertension (rHT), or other hypertension related diseases with an ERA is considered.

The ET_{A}-selective endothelin receptor antagonist darusentan has been in development for the treatment of resistant hypertension (rHT) (Bakris et al., Hypertension 2010, 56,824-830, see also WO2007/098390). In a 14 week phase 3 trial in patients with rHT, it demonstrated efficacy on the reduction of ambulatory blood pressure, but failed to show significant treatment effect on the primary endpoint systolic blood pressure. Patients were eligible to participate if they had treatment resistant hypertension (systolic blood pressure of higher than 140 mm Hg) despite treatment with three or more antihypertensive drugs from different drug classes, including a diuretic, at optimized doses. A minimum dose of 25 mg per day of hydrochlorothiazide (or its equivalent for other thiazide diuretic drugs) was required. Even though during the trial diuretic therapy could be intensified at the discretion of the investigators to manage fluid retention, the most frequent adverse event associated with darusentan was fluid retention/edema at 28% versus 12% in each of the other groups. More patients withdrew because of adverse events on darusentan as compared with placebo.

The ET_{A}-selective ERA avosentan, in a trial that investigated the effects of avosentan on progression of overt diabetic nephropathy in patients with type 2 diabetes, showed significant treatment effect, associated with a significantly increased discontinuation of trial medications due to adverse events, predominantly related to fluid overload and congestive heart failure (Mann et al., “Avosentan for Overt Diabetic Nephropathy”, J Am Soc Nephrol. 2010, 21(3): 527-535.). The composite primary outcome was the time to doubling of serum creatinine, ESRD, or death. Secondary outcomes included changes in albumin-to-creatinine ratio (ACR) and cardiovascular outcomes. The study did not detect a difference in the frequency of the primary outcome between groups. Avosentan significantly reduced ACR. The trial was terminated prematurely after a median follow-up of 4 months (maximum 16 months) because of an excess of cardiovascular events with avosentan, and the authors conclude that “it may be that at dosages of 25 to 50 mg avosentan is less selective for the ET_{A} receptor and thus caused sodium and water retention and peripheral vasodilation with a potential fluid shift from the intravascular to extravascular space”. The effect on albuminuria was considered likely due to inhibition of the renal ET_{A} receptor, because it was previously found that the mixed type ET_{A/B} receptor antagonists have a weaker or no effect on proteinuria. According to the authors, the assumption of ET_{B} receptor blockade with higher dosages of avosentan is further supported by data that showed a natriuretic effect of selective ET_{A} receptor blockade in people who were treated with ACE inhibitors. Thus, the natriuretic effect/fluid retention that possibly in final consequence lead to the discontinuation of the trial was attributed to a dual blockade of the ET_{A} and the ET_{B} receptor, discouraging from using a dual acting ERA in such clinical setting.

Further pre-clinical data showed that the synergistic effect on blood pressure of an ET_{A}-selective ERA in combination with the ACE inhibitor enalapril was abolished by simultaneous blockade of the ET_{B}-receptor (Goddard et al., J.Am.Soc.Nephrol. 2004, 15, 2601-2610), thus, discouraging from using a dual acting ERA in a clinical setting where ACE inhibitors may be required as background therapy.

In a review on “Endothelin antagonists for diabetic and non-diabetic chronic kidney disease” (Br J Clin Pharmacol (2012), 76:4, 573-579), D. E. Kohan et al. state that “in general, the prevailing opinion is that ET_{A}, as opposed to combined ET_{A/B}, receptor antagonists are preferred for treating CKD”. Three years later Kohan et al. conclude with regard to a study published in Clin J Am Soc Nephrol (2015), 10:1568-1574 that “the fluid-retaining effect of ERAs is most likely related to direct effects on renal tubular sodium transport, whereas the antiproteinuric effect of ERAs is likely associated with actions on the vasculature and/or glomerulus. Finally, it could be anticipated that ERA mitigation of proteinuria per se would favor renal fluid excretion; however, ERAs could still promote fluid retention through a separate effect on tubule sodium and water reabsorption”.

WO2016/073846 provides a comprehensive summary of ERAs tested for various indications including diabetic and non-diabetic CKD and rHT. WO2016/073846 provides further examples where fluid retention may have led to increased side effects for the ERAs bosentan, tezosentan, ambrisentan, and atrasentan. WO2016/073846 concludes in proposing a method of treating CKD with an ERA, especially with the ET_{A}-selective ERA atrasentan, using predictors of fluid retention; said method comprising the determination of a risk of fluid retention if an ERA were administered to the subject; and administering the ERA to the subject if the risk is at an acceptable level. The detailed study protocol of a clinical phase 3 study (SONAR) evaluating the effects of the investigational compound atrasentan—when added to standard of care-on progression of kidney disease in patients with stage 2 to 4 chronic kidney disease and type 2 diabetes was published in Heerspink et al, Diabetes Obes. Metab. 2018, 1-8. The protocol reflects the importance given to dose optimization and simultaneous control of sodium retention/fluid retention in the study design, leading to a study design that requires “the selection of individuals at high risk of disease (prognostic enrichment) who also demonstrate a good response to study treatment (predictive enrichment)”. However, on Dec. 1, 2017, AbbVie announced its strategic decision to close the SONAR study. The press release states that “the ongoing monitoring of renal events observed in the study has revealed considerably fewer end-points than expected by this time, which will likely affect the ability to test the SONAR study hypothesis. Therefore, AbbVie has determined that it cannot justify continuing the participation of patients in the study. The decision to close the SONAR study early was not related to any safety concerns.”

Contrary to the conclusions drawn from the avosentan trial, preclinical and clinical data suggest that the ET_{A}-selective antagonists sitaxentan and ambrisentan pose a greater risk of fluid retention than the dual ERAs bosentan and macitentan (Vercauteren et al., JPET 2017, 361, 322-333). The authors state that their findings “indicate that in rats, stimulation of the unblocked ET_{B} receptors in presence of ET_{A} receptor antagonist, but not functional antagonism of the ET_{A} receptor per se, can be detrimental, and that blockade of both receptors is less likely to cause water retention than single receptor blockade” and continue to speculate that “plasma volume expansion combined with increased vascular permeability could explain the observations obtained with ET_{A}-selective antagonists”. The authors conclude that “several clinical studies with ET_{A}-selective antagonists have resulted in mortality increases in relation to fluid retention issues, whereas this has not been observed with dual ERAs. Dual ERAs, however, in conditions of preexisting fluid retention or arginine vasopressin (AVP) increase, such as chronic heart failure or chronic renal failure, have caused significant fluid retention”.

It has been shown in a phase 2 clinical trial that aprocitentan, an ERA resulting in effective dual blockade of the endothelin receptors, may result in efficacious control of blood pressure in subjects having essential hypertension (aprocitentan was administered as monotherapy, i.e. without background anti-hypertensive therapy) (Actelion Pharmaceuticals Ltd, press release May 22, 2017; P. Verweij et al. 2020: https://www.ahajournals.org/doi/full/10.1161/HYPERTENSIONAHA.119.14504). The study evaluated the efficacy, safety and tolerability of a once-a-day oral regimen of 4 dose levels of aprocitentan (5, 10, 25, and 50 mg) to identify the optimal doses for further studies. In this study, 490 patients were randomized to receive either aprocitentan 5, 10, 25, 50 mg, placebo, or lisinopril 20 mg once daily. After 8 weeks of treatment the mean reduction from baseline in diastolic blood pressure-as measured at trough with a novel automated office blood pressure device-ranged between 6.3 and 12.0 mmHg in a statistically significant dose-dependent manner for the aprocitentan groups versus a decrease of 4.9 mmHg in the placebo group and a decrease of 8.4 mmHg in the lisinopril group (in the per-protocol population comprised of 410 patients). Systolic blood pressure reductions ranged from 10.3 to 18.5 mmHg in a statistically significant dose-dependent manner in the aprocitentan groups and were 7.7 and 12.8 mmHg in the placebo and lisinopril groups, respectively. These findings were confirmed in all randomized patients (Intent-to-Treat principle) and by 24 hours Ambulatory Blood Pressure Monitoring. The safety population included 327 patients in the aprocitentan groups, 82 patients in the placebo group and 81 in the lisinopril group. Aprocitentan was well tolerated across all four doses in this patient population. Discontinuation from study treatment due to an adverse event ranged between 1.2% and 3.7% for the aprocitentan groups versus 6.1% in the placebo group and 3.7% in the lisinopril group. The overall frequency of adverse events was similar to those observed in the placebo group. There were two cases of increased liver enzymes above three times the upper limit of the normal range, one in the placebo and one in the aprocitentan 5 mg group. Four cases of peripheral edema were observed, two in the aprocitentan 25 mg group and two in the aprocitentan 50 mg group. Mean body weight remained unchanged from baseline in the aprocitentan 5, and mg groups, increased by 0.4 kg in the aprocitentan 25 and 50 mg groups, and by 0.3 kg in the placebo group 10 and decreased by 0.3 kg on lisinopril. There was an expected dose related decrease from baseline in the hemoglobin concentration (an indicator of haemodilution) in the aprocitentan groups (ranging from 1.3 to 6.7 g/L) versus increases of 2.2 and 0.1 g/L in the placebo and lisinopril groups, respectively (see also P. Gueneau de Mussy et al; Clin Pharm & Therapeutics 2020; doi: 10.1002/cpt.2043).

Thus, different from the methods of WO2016/073846 no risk assessment and/or dose reduction to mitigate side effects related to fluid retention may be required for aprocitentan when used in the treatment of hypertension related diseases, especially resistant hypertension. Thus, aprocitentan may have a different pharmacological profile than the predominantly ET_{A}-selective antagonists so far tested in resistant hypertension or chronic kidney disease in diabetic and non-diabetic patients. Following the positive phase 2 clinical trial results, aprocitentan has been advanced into a phase 3 clinical trial (NCT03541174): “A Research Study to Show the Effect of Aprocitentan in the Treatment of Difficult to Control (Resistant) High Blood Pressure (Hypertension) and Find Out More About Its Safety” (see also Danaietash P et al; Identifying and treating resistant hypertension in PRECISION—a randomized long-term clinical trial with Aprocitentan. J Clin Hypertension 2022, 24(7):804-813; https://doi.org/10.1111/jch.14517; M. Clozel, Aprocitentan and the endothelin system in resistant hypertension; Can. J. Physiol. Pharmacol. 2022; https://doi.org/10.1139/cjpp-2022-0010; both documents are incorporated by reference). Results of the PRECISION trial were announced in a media release by Idorsia Pharmaceuticals on May 23 2022 (https://www.idorsia.com/media/news-details?newsId=2758691). Subsequently the results were published Nov. 7, 2022 in a peer reviewed journal (Schlaich et al., Lancet 2022; 400:1927-37 and associated supplementary material, both herewith incorporated by reference), the publication being covered by a media release (https://www.idorsia.com/media/news-details?newsId=2869821) and a webcast presentation on Nov. 8, 2022.

Moreover, it has been found in rat models of hypertension that aprocitentan may have synergistic pharmacological effect in combination with angiotensin receptor blockers (ARBs) such as valsartan, may in certain models have synergistic pharmacological effect in combination with angiotensin converting enzyme (ACE) inhibitors such as enalapril, and may have additive pharmacological effect in combination with calcium channel blockers (CCBs) such as amlodipine (F. Trensz et al. 2019: https://doi.org/10.1124/jpet.118.253864).

In particular, when combined with three antihypertensive therapies of different pharmacological classes including valsartan, amlodipine, and a diuretic of the thiazide class such as commercially available Exforge HCT® (i.e. a fixed dose combination of valsartan/amlodipine/hydrochlorothiazide), aprocitentan may result in superior effect than for example spironolactone which is a standard available add-on treatment. Moreover, aprocitentan may have a different pharmacological profile than the predominantly ET_{A}-selective antagonists so far tested in resistant hypertension and other endothelin-related diseases. Thus, aprocitentan, an ERA resulting in effective dual blockade of the endothelin receptors, may be particularly suited for the treatment of (resistant) hypertension when prescribed in combination with standard background therapy, generally including one or more antihypertensive therapies of different pharmacological classes, including especially an angiotensin receptor blocker such as valsartan, a calcium channel blocker such as amlodipine, and/or a diuretic, especially a diuretic of the thiazide class (a thiazide-like diuretic) such as chlorothiazide, chlorthalidone, hydrochlorothiazide, indapamide, or metolazone (WO2018/153513, WO2018/154101). Such combination treatment may result in superior control of blood pressure compared to the treatment with such antihypertensive therapies alone, while maintaining a benign side effect profile even at optimal efficacious dosages of aprocitentan, not requiring e.g. the risk assessment methods of WO2016/073846 and/or dose reductions to mitigate side effects, e.g. related to fluid retention.

Further standard background therapy, in particular for the treatment of a patient having a history of hypertension, comprises beta blockers (beta-adrenergic blocking agents, blocking the effects of the hormone epinephrine (adrenaline). Beta blockers cause the heart to beat more slowly and with less force, which lowers blood pressure, and help widen veins and arteries to improve blood flow.

Further standard background therapy, in particular for the treatment of a patient having a history of diabetes or diabetic kidney disease, or in patients having patients having established cardivascular disease, comprises SGLT-2 inhibitors such as atigliflozin, bexagliflozin, canagliflozin, dapagliflozin, empagliflozin, ertugliflozin, henagliflozin, ipragliflozin, luseogliflozin, remogliflozin, sotagliflozin, tianagliflozin, or tofogliflozin block glucose reabsorption in the kidney, increase glucose excretion, and lower blood glucose concentration. In addition to this well characterized mode of action, SGLT-2 inhibitors reduce blood pressure, decrease vascular stiffness, improve endothelial function, and have anti-inflammatory and anti-fibrotic properties resembling those of ERAs (H. J. Heerspink et al., Circulation (2016), 134(10): 752-772). This unique mechanism of action lead to the development and market approval of several SGLT-2 inhibitors comprising canagliflozin, dapagliflozin and empagliflozin, all indicated to improve glycemic control in adults with type 2 diabetes mellitus, empagliflozin in addition being indicated to reduce the risk of cardiovascular death in such patients having established cardivascular disease. Sotagliflozin, a dual SGLT-1 and SGLT-2 inhibitor has been reported to be in clinical trials for type 1 diabetes.

Diabetes ofter concurs with heart failure (HF) and may contribute to its development. SGLT-2 inhibitors such as empagliflozin may be suitable for the treatment of chronic HF, including especially also HFpEF where treatment options are very limited. The EMPA-REG OUTCOME Trial (Empagliflozin, Cardiovascular Outcomes, and Mortality in Type 2 Diabetes) randomized type II diabetic patients with high cardivascular risk to empaglilflozin or standard of care. The results suggested improvement in cardiovascular death, non-fatal myocardial infarction, nonfatal stroke, hospitalization for HF, and death from any cause. A post hoc study looking at patients with a diagnosis of HF at baseline suggested significantly lowered cardiovascular death, HF hospitalization, and all cause hospitalization (D. H. Kim et al., “Pharmacologic Management for Heart Failure and Emerging Therapies” Curr Cardiol. Rep (2017) 19:94). The mode of action of SGLT-2 leads to simultaneous inhibition of glucose and sodium uptake in the proximal tubules of the nephron, believed to result in a reset of the tubulo-glomerular feedback putatively causing the phenomenon of glomerular hyperfiltration. Efficacy of SGLT-2 inhibitors is believed to decrease with lower plasma glucose levels or a drop in glomerular filtration rate (GFR), thus, SGLT-2 inhibitors have an inherent low risk for developping hypoglycemia. In consequence, the properties of SGLT-2 inhibitors may open a pathway to treat HF including HFpEF even in non-diabetic patients (P. Martens et al., “Promise of SGLT2 Inhibitors in Heart Failure: Diabetes and Beyond”, Curr Treat Options Cardio Med (2017) 19:23). A side effect associated with the pharmacological effects of SGLT-2 inhibitors may be volume depletion/intravascular volume contraction, potentially leading to dehydration, hypovolemia, orthostatic hypotension, or hypotension. Thus, SGLT-2 inhibitors generally may induce an increase in hematocrit (Hct), a marker of haemoconcentration and increased blood viscosity, a putative cause of vascular injury in a context of peripheral vascular disease. Furthermore, data from large clinical trials suggest that SGLT2 inhibitors may induce acute kidney injury and impairment in renal function, especially in patients predisposed to acute kidney injury where hypovolemia, chronic renal insufficiency, congestive heart failure and concomitant medications (diuretics, ACE inhibitors, ARBs and NSAIDs) are to be considered. The pharmacological action of SGLT-2 inhibitors on the kidney includes an increase of serum creatinine and a decrease eGFR.

The combination of aprocitentan with SGLT-2 inhibitors may be of particular interest (WO2019/106066). Preliminary data obtained from the above-referenced PRECISION trial indicated that the available data, taking into consideration the small available sample size, can be considered: (a) to confirm the pronounced effect of the treatment of aprocitentan on blood pressure data points (clinical endpoints of the study), whether alone on top of standardized antihypertensive background therapy, or in subjects who received as background therapy an SGLT-2 inhibitor and said standardized antihypertensive background therapy; and (b) to indicate (i) an improved antiproteinuric effect relevant for the kidney diseases CKD/DKD for the present combination treatment; and (ii) a smaller decrease of hemoglobin value from baseline to week 36, indicating a further potential clinical benefit of the present combination treatment.

Hypertension is one of the most common cardiovascular risk factors, and its prevalence continues to rise. According to a recent study, there are more than one billion people living with hypertension worldwide—a number which has almost doubled in the past 40 years. [Bin Zhou, et al. The Lancet; 2017; 389(10064):37-55]. While many patients with hypertension are successfully treated with various existing anti-hypertensive therapies, 10-20% of the hypertensive population have blood pressure which that remains high despite receiving at least three antihypertensive medications of different pharmacological classes, including a diuretic, at optimal doses, and they are categorized in hypertension guidelines [R. M. Carey, et al. Hypertension, 2018; 72, pp. e53-e90; Noubiap, J. J., et al., Heart 2019; 105:98-105; Carey RM, et al. Hypertension. 2019; 73(2): 424-431] and in the medical community as having resistant hypertension. Certain populations are at a particular high risk of developing resistant hypertension later in life; these include patients with a high body mass index (BMI), African Americans, post-menopausal women and patients with obstructive sleep apnea. [Coylewright, M., et al. Hypertension, 2008; 51, 952-9; Roberie, D. R., et al. Curr Opin Cardiol, 2012; 27, 386-91; Khan, A., et al. Int J Hypertens, 2013; 193010-193010]. It is estimated that by 2025, there could be approximately 10 million patients in the US who could be classified as having resistant hypertension and a similar number of patients in Europe. [Noubiap, J. J., et al., Heart 2019; 105:98-105; Carey R M, et al. Hypertension. 2019; 73(2):424-431; Lu Y, et al. Hypertension. 2022; 79(1):207-217]. Uncontrolled hypertension can lead to multiple cardiovascular and renal adverse outcomes, including stroke, heart disease, and kidney failure. These co-morbidities increase a patient's vulnerability and the complexity of their treatment. [Daugherty, S. L., et al. Circulation. 2012; 125(13):1635-42; Kumbhani, D. J., et al. Eur Heart J. 2013; 34(16):1204-14; Muntner, P., et al. Hypertension. 2014; 64:1012-1021]. The current direction in the management of hypertension is toward earlier and lower BP control for 24 hours, including the nocturnal and morning periods. The night-time blood pressure management is especially important to prevent cardiovascular events, especially heart failure (K. Kario, Hypertension. 2018;71:997-1009). E. Dolan et al. found that hazard ratios for nighttime ambulatory blood pressure remained significant after adjustment for daytime ambulatory blood pressure. From these results the authors conclude for two important clinical messages: ambulatory measurement of blood pressure is superior to clinic measurement in predicting cardiovascular mortality, and nighttime blood pressure is the most potent predictor of outcome (E Dolan et al, Hypertension 2005, 46(1): 156-161).

According to K. Kario, Hypertension. 2018;71:997-1009, herewith incorporated by reference, the pattern of circadian rhythm of BP can for example be evaluated by ambulatory BP monitoring (ABPM). In healthy subjects, night-time BP decreases by 10% to 20% of daytime BP (normal dipper pattern). This circadian rhythm of BP is determined partly by the intrinsic rhythm of central and peripheral clock genes, which regulate the neurohumoral factor and cardiovascular systems, and partly by the sleep-wake behavioral pattern. Hypertensive patients without organ damage also exhibit the dipper pattern; however, those with organ damage tend to exhibit nondipper patterns with diminished night-time BP fall (or even riser patterns where night-time BP is higher than day-time BP). Night-time BP dipping patterns are classified into four groups: riser, nondipper, dipper, and extreme dipper patterns. The definitions of these groups are based on night-time BP dipping. Kario states that the nocturnal hypertension and nondipper/riser patterns of night-time BP are predisposing conditions for psychocognitive dysfunction (cognitive dysfunction, apathy, falls and sedentary lifestyle, and stroke), hypertensive heart disease (left ventricular hypertrophy, reduced diastolic function), vascular damage (increase in carotid intima-media thickness, pulse wave velocity, and cardio ankle vascular index), and chronic kidney disease (CKD; reduced glomerular filtration ratio and urinary albumin/creatinine excretion ratio).

Burnier and Damianaki state that, in patients with CKD, reduced or reverse dipping patterns or masked and resistant hypertension are frequent and associated with a poor cardiovascular and renal prognosis. Current antihypertensive options have been enriched with novel agents that enable to lower the existing renal and cardiovascular risks, such as SGLT-2 inhibitors and novel nonsteroidal mineralocorticoid receptor antagonists. (Burnier and Damianaki; Circulation Research. 2023;132:1050-1063; herewith included by reference). The authors conclude: “Hypertension is a major cardiovascular risk factor in the general population but even more so in patients with CKD, who cumulate several other risk factors including the reduced kidney function. Patients with CKD are characterized by several specific BP profiles and hypertension phenotypes that deserve to be diagnosed accurately to avoid misdiagnoses. To this purpose, out-of-office BP measurements that include also the nighttime period are now strongly recommended and should be used more widely to verify that BP is under control during the day as well as during the night. Today, a high percentage of patients with CKD have a poorly controlled BP, mainly because nighttime BP is elevated.”

Thus, aprocitentan, an ERA resulting in effective dual blockade of the endothelin receptors, may be particularly suited for a clinically proven effective and safe long-term treatment of (chronic) hypertension, in particular (chronic) resistant hypertension, wherein it is understood that treatment of resistant hypertension generally comprises combination with standard background therapies; e.g. modulators of the renin-angiotensin system such as especially ARBs or ACE inhibitors; CCBs; diuretics; and/or beta blockers. It is further understood that the patient population being diagnosed with such difficult to control or resistant hypertension is a frail patient population generally having one or more comorbidities especially comprising diabetes mellitus, ischemic heart disease, stroke, congestive heart failure and/or sleep aponea syndrome. In addition, a further comorbidity (associated or not with the above-listed comorbidities) may be CKD (e.g. CKD, or CKD associated with diabetes (DKD)). Therefore, (additional) background therapy, such as especially SGLT-2 inhibitors, may be of particular interest in combination with a clinically proven safe and clinically proven effective antihypertensive of a different class, such as aprocitentan, especially in case of certain comorbidities including notably diabetes mellitus, ischemic heart disease, congestive heart failure and/or CKD (including CKD associated with diabetes (DKD). It has been found that aprocitentan or a pharmaceutically acceptable salt thereof is particularly useful to provide a proven clinically effective treatment for certain endothelin related disorders, especially hypertension including difficult to control and resistant hypertension, that require significant and long-term sustained decrease of systolic and diastolic blood pressure, in particular when used in subjects presenting one or more co-morbidities, and in combination with other active ingredients or therapeutic agents that are standard background therapies for such hypertension disorders, or standard background therapies for diseases or disorders generally associated with hypertension.

### DETAILED DESCRIPTION OF THE INVENTION

1) A first embodiment of the invention relates to a method of treating endothelin related disorders comprising especially hypertension including difficult to control and resistant hypertension; chronic kidney disease (CKD) [notably CKD of stages 1 to 4 as defined by the Kidney Disease Improving Global Outcomes (KDIGO) Guidelines (and especially CKD of stage 3 or 4)], and in particular CKD (notably of these stages) caused by/associated with hypertension and/or caused by/associated with diabetes (diabetic kidney disease (DKD)); or diabetes; in a subject in need thereof comprising administering to the subject a pharmaceutical composition comprising a clinically proven effective amount of aprocitentan, or a pharmaceutically acceptable salt thereof.

In particular, such CKD herein-above may be associated with macroproteinuria (defined as UACR >300 mg/g). UACR is a biomarker of renal dysfunction, which is monitored in renally impaired patients (Levey et al., Uses of GFR and albuminuria level in acute and chronic kidney disease. N Engl J Med. 2022;386(22):2120-28].

It is understood that such method of treating CKD (notably CKD of stage 3 or 4) caused by/associated with hypertension and optionally further associated, in addition, with diabetes (diabetic kidney disease (DKD)) likewise refers to treating hypertension including difficult to control and resistant hypertension, wherein said hypertension includes said hypertension causing/associated with such CKD, and wherein said CKD optionally is further associated, in addition to said hypertension, with diabetes.
2) A further embodiment relates to the method according to embodiment 1), wherein said subject in need thereof is a subject receiving standard background therapy (wherein it is understood that such standard background therapy is suitable for the treatment of hypertension).
3) A further embodiment relates to the method according to embodiment 2) wherein said standard background therapy comprises first line therapies suitable for the prevention or treatment of hypertension, such as especially an angiotenin receptor blocker (ARB) or an ACE inhibitor as hypertension treatment, notably (further) in combination with a calcium channel blocker (CCB) and/or a diuretic (especially a thiazide-like diuretic); and optionally (further) in combination with a beta blocker.

It is understood that preferably such standard background therapy comprises, if present, only one active ingredient that is a modulator of the renin-angiotensin system, i.e. either an ARB or an ACE inhibitor. It is further understood that beta blockers are a recommended standard background therapy for subjects having a medical history of cardiac comorbidities such as especially ischemic heart disease, and/or congestive heart failure
4) A further embodiment relates to the method of embodiment 2) wherein said standard background therapy comprises:
   an angiotenin receptor blocker (especially valsartan, as well as losartan, candesartan, irbesartan, telmisartan, eprosartan, olmesartan, azilsartan, fimasartan), or a pharmaceutically acceptable salt thereof; or
      an ACE inhibitor (especially enalapril, ramipril, quinapril, perindopril, lisinopril, benazepril, imidapril, trandolapril, cilazapril), or a pharmaceutically acceptable salt thereof; and/or
   a calcium channel blocker (especially amlodipine, as well as aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, pranidipine), or a pharmaceutically acceptable salt thereof; and/or
   a diuretic including loop diuretics including furosemide, bumetanide, ethacrynic acid, torsemide; potassium-sparing diuretics including aldosterone antagonists such as spironolactone, eplerenone, or finerenone, or aldosterone synthase inhibitors; carbonic anhydrase inhibitors including acetazolamide and methazolamide; in particular diuretics of the thiazide class (thiazide-like diuretics) such as especially chlorthalidone, hydrochlorothiazide, chlorothiazide, indapamide, or metolazone (preferred diuretics are thiazide-like diuretics, especially chlorthalidone or hydrochlorothiazide); and/or
   a beta blocker (beta-adrenergic blocking agent), especially acebutolol, atenolol, bisoprolol, metoprolol (immediate release or sustained release), nadolol, nebivolol, propranolol (immediate release or sustained release);

wherein it is understood that such standard background therapy may comprise at least one (especially one to four) of the above-listed standard background therapy agents and, in addition, further background therapy agents of the above-listed wherein such further background therapy agents is of different pharmacological action, such as for example an additional diuretic of different pharmacological action.
4a) A first sub-embodiment of this embodiment 4), thus, relates to the method of embodiment 2), wherein said standard background therapy comprises [wherein notably said standard background therapy comprises at least two (especially comprises three or four) other antihypertensive drugs selected from the group consisting of]:
   an angiotenin receptor blocker (especially valsartan, as well as losartan, candesartan, irbesartan, telmisartan, eprosartan, olmesartan, azilsartan, fimasartan), or a pharmaceutically acceptable salt thereof; or
      an ACE inhibitor (especially enalapril, ramipril, quinapril, perindopril, lisinopril, benazepril, imidapril, trandolapril, cilazapril), or a pharmaceutically acceptable salt thereof; and/or
   a calcium channel blocker (especially amlodipine, as well as aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, pranidipine), or a pharmaceutically acceptable salt thereof; and/or
   an aldosterone antagonist such as spironolactone, eplerenone, or finerenone; or an aldosterone synthase inhibitor (such as baxdrostat); and/or
   a diuretic selected from:
      loop diuretics including furosemide, bumetanide, ethacrynic acid, or torsemide;
      carbonic anhydrase inhibitors including acetazolamide or methazolamide;
      in particular diuretics of the thiazide class (thiazide-like diuretics) such as especially chlorthalidone, hydrochlorothiazide, chlorothiazide, indapamide, or metolazone (preferred diuretics are thiazide-like diuretics, especially chlorthalidone or hydrochlorothiazide); or
      potassium sparing diuretics which are not aldosterone antagonists or aldosterone synthase inhibitors (an example is amiloride);
   a beta blocker (beta-adrenergic blocking agent), especially acebutolol, atenolol, bisoprolol, metoprolol (immediate release or sustained release), nadolol, nebivolol, propranolol (immediate release or sustained release).
4b) A second sub-embodiment of this embodiment 4), thus, relates to the method of embodiment 2), wherein said standard background therapy comprises [wherein notably said standard background therapy comprises at least two (especially comprises three or four) other antihypertensive drugs selected from the group consisting of]:
   an angiotenin receptor blocker (especially valsartan, as well as losartan, candesartan, irbesartan, telmisartan, eprosartan, olmesartan, azilsartan, fimasartan), or a pharmaceutically acceptable salt thereof; or
      an ACE inhibitor (especially enalapril, ramipril, quinapril, perindopril, lisinopril, benazepril, imidapril, trandolapril, cilazapril), or a pharmaceutically acceptable salt thereof; and/or
   a calcium channel blocker (especially amlodipine, as well as aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, pranidipine), or a pharmaceutically acceptable salt thereof; and/or
   a loop diuretic including furosemide, bumetanide, ethacrynic acid, torsemide; and/or
   a further diuretic selected from
      potassium sparing diuretics which are not aldosterone antagonists or aldosterone synthase inhibitors, such as amiloride;
      aldosterone antagonists such as spironolactone, eplerenone, or finerenone; or aldosterone synthase inhibitors (such as baxdrostat);
      carbonic anhydrase inhibitors including acetazolamide and methazolamide; or
      in particular diuretics of the thiazide class (thiazide-like diuretics) such as especially chlorthalidone, hydrochlorothiazide, chlorothiazide, indapamide, or metolazone (preferred diuretics are thiazide-like diuretics, especially chlorthalidone or hydrochlorothiazide); and/or
   a beta blocker (beta-adrenergic blocking agent), especially acebutolol, atenolol, bisoprolol, metoprolol (immediate release or sustained release), nadolol, nebivolol, propranolol (immediate release or sustained release).
5) A further embodiment relates to the method according to any one of embodiments 1) to 3), wherein said subject in need thereof is a subject receiving at least three other antihypertensive drugs independently selected from the group consisting of
   an angiotenin receptor blocker (especially valsartan, as well as losartan, candesartan, irbesartan, telmisartan, eprosartan, olmesartan, azilsartan, fimasartan), or a pharmaceutically acceptable salt thereof;
   an ACE inhibitor (especially enalapril, ramipril, quinapril, perindopril, lisinopril, benazepril, imidapril, trandolapril, cilazapril), or a pharmaceutically acceptable salt thereof;
   a calcium channel blocker (especially amlodipine, as well as aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, pranidipine), or a pharmaceutically acceptable salt thereof;
   a diuretic including loop diuretics including furosemide, bumetanide, ethacrynic acid, torsemide; potassium-sparing diuretics including aldosterone antagonists such as spironolactone, eplerenone, or finerenone, or aldosterone synthase inhibitors; carbonic anhydrase inhibitors including acetazolamide and methazolamide; in particular diuretics of the thiazide class (thiazide-like diuretics) such as especially chlorthalidone, hydrochlorothiazide, chlorothiazide, indapamide, or metolazone (preferred diuretics are thiazide-like diuretics, especially chlorthalidone or hydrochlorothiazide); and
   a beta blocker (beta-adrenergic blocking agent), especially acebutolol, atenolol, bisoprolol, metoprolol (immediate release or sustained release), nadolol, nebivolol, propranolol (immediate release or sustained release);

wherein it is understood that preferably said at least three other antihypertensive drugs comprise, if present, only one modulator of the renin-angiotensin system, i.e. either an ARB or an ACE inhibitor.
6) A further embodiment relates to the method according to any one of embodiments 1) to 3), wherein aprocitentan is administered in combination with at least three other antihypertensive drugs independently selected from the group consisting of
   an angiotenin receptor blocker (especially valsartan, as well as losartan, candesartan, irbesartan, telmisartan, eprosartan, olmesartan, azilsartan, fimasartan), or a pharmaceutically acceptable salt thereof; or
      an ACE inhibitor (especially enalapril, ramipril, quinapril, perindopril, lisinopril, benazepril, imidapril, trandolapril, cilazapril), or a pharmaceutically acceptable salt thereof;
   a calcium channel blocker (especially amlodipine, as well as aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, pranidipine), or a pharmaceutically acceptable salt thereof;
   a diuretic including loop diuretics including furosemide, bumetanide, ethacrynic acid, torsemide; potassium-sparing diuretics including aldosterone antagonists such as spironolactone, eplerenone, or finerenone, or aldosterone synthase inhibitors; carbonic anhydrase inhibitors including acetazolamide and methazolamide; in particular diuretics of the thiazide class (thiazide-like diuretics) such as especially chlorthalidone, hydrochlorothiazide, chlorothiazide, indapamide, or metolazone (preferred diuretics are thiazide-like diuretics, especially chlorthalidone or hydrochlorothiazide); and
   a beta blocker (beta-adrenergic blocking agent), especially acebutolol, atenolol, bisoprolol, metoprolol (immediate release or sustained release), nadolol, nebivolol, propranolol (immediate release or sustained release);

wherein especially one modulator of the renin-angiotensin system selected from an ARB and an ACE inhibitor is administered in such combination treatment; wherein it is understood that only one modulator of the renin-angiotensin system, i.e. either an ARB or an ACE inhibitor is administered.
7) A further embodiment relates to the method according to any one of embodiments 1) to 6), wherein said subject has a medical history of comorbidities comprising diabetes (especially diabetes mellitus), ischemic heart disease, stroke, and/or congestive heart failure.
8) A further embodiment relates to the method according to any one of embodiments 1) to 7), wherein, in case said subject has a medical history of diabetes (especially diabetes mellitus), said subject is a subject receiving (in addition to standard background therapy suitable for the treatment of hypertension) a standard background therapy suitable.
   to reduce the risk of cardiovascular death and hospitalization for heart failure in adults with heart failure, and/or.
   to reduce the risk of cardiovascular death in adults with type 2 diabetes mellitus and established cardiovascular disease; and/or
   as an adjunct to diet and exercise to improve glycemic control in adults with type 2 diabetes mellitus; and/or
   for the treatment of diabetes (in particular type 2 diabetes);

wherein especially said subject is a subject receiving (in addition to standard background therapy suitable for the treatment of hypertension) a standard background therapy suitable for the treatment of diabetes (in particular type 2 diabetes).
9) A further embodiment relates to the method according to any one of embodiments 1) to 8), wherein said subject is a subject receiving, in addition to standard hypertension background therapy, further standard background therapy, notably suitable for the treatment of diabetes; wherein said additional standard background therapy comprises
   an SGLT-2 inhibitor (especially atigliflozin, bexagliflozin, canagliflozin, dapagliflozin, empagliflozin, ertugliflozin, henagliflozin, ipragliflozin, luseogliflozin, remogliflozin, sotagliflozin, or tofogliflozin; or a pharmaceutically acceptable salt thereof; especially bexagliflozin, canagliflozin, dapagliflozin, empagliflozin, ertugliflozin, henagliflozin, ipragliflozin, luseogliflozin, sotagliflozin, or tofogliflozin; in particular canagliflozin, or dapagliflozin, or empagliflozin), or a pharmaceutically acceptable salt thereof; and/or
   metformin; and/or
   insulin; and/or
   a sulfonylurea (especially glibenclamide), or a pharmaceutically acceptable salt thereof; and/or
   a DPP-4 inhibitor (especially sitagliptin, vildagliptin, saxagliptin, or linagliptin), or a pharmaceutically acceptable salt thereof; and/or
   a GLP-1 receptor agonist (especially exenatide, liraglutide, lixisenatide, albiglutide, dulaglutide, taspoglutide, semaglutide; or a dual GLP-1/GIP agonist such as trizepatide); and/or
   a thiazolidinedione, or a pharmaceutically acceptable salt thereof;

wherein in particular such additional standard background therapy comprises an SGLT-2 inhibitor; metformin, and/or a DPP-4 inhibitor;

in particular an SGLT-2 inhibitor, wherein said SGLT-2 inhibitor (notably bexagliflozin, canagliflozin, dapagliflozin, empagliflozin, ertugliflozin, henagliflozin, ipragliflozin, luseogliflozin, sotagliflozin, or tofogliflozin;

in particular canagliflozin, or dapagliflozin, or empagliflozin) is especially indicated.
to reduce the risk of cardiovascular death and hospitalization for heart failure in adults with heart failure, and/or.
   to reduce the risk of cardiovascular death in adults with type 2 diabetes mellitus and established cardiovascular disease; and/or
   as an adjunct to diet and exercise to improve glycemic control in adults with type 2 diabetes mellitus.

It is understood that such method according to any one embodiments 1) to 9) likewise refers to aprocitentan, or a pharmaceutically acceptable salt thereof, for use in the treatment of hypertension including difficult to control hypertension and resistant hypertension, wherein aprocitentan is to be administered in combination with such standard background therapy.

It is further understood that such method according to any one embodiments 1) to 9) likewise refers to a pharmaceutical composition comprising aprocitentan, or a pharmaceutically acceptable salt thereof, wherein said pharmaceutical composition is for use in such method, especially for use in the treatment of hypertension including difficult to control hypertension and resistant hypertension, or chronic kidney disease (CKD) [notably CKD of stages 1 to 4 as defined by the Kidney Disease Improving Global Outcomes (KDIGO) Guidelines (and especially CKD of stage 3 or 4)], and in particular CKD (notably of these stages) caused by/associated with hypertension and/or caused by/associated with diabetes (diabetic kidney disease (DKD)); wherein said pharmaceutical composition
comprises, as further active ingredient(s), such standard background therapy/therapies; or
is to be administered in combination with such standard background therapy/therapies.

The term “Angiotensin Receptor Blocker” or “ARB” in particular means in the present application valsartan, losartan, telmisartan, irbesartan, candesartan, olmesartan, azilsartan, or a pharmaceutically acceptable salt of one of these. A preferred ARB is valsartan or a pharmaceutically acceptable salt thereof.

The term “Angiotensin Converting Enzyme inhibitor” or “ACE inhibitor” in particular means in the present application captopril, enalapril, ramipril, quinapril, perindopril, lisinopril, imidapril or cilazapril, or a pharmaceutically acceptable salt of one of these.

The term “Calcium Channel Blocker” or “CCB” in particular means in the present application amlodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nimodipine, nisoldipine, nitrendipine, pranidipine, verapamil or diltiazem or a pharmaceutically acceptable salt of one of these. A preferred CCB is amlodipine or a pharmaceutically acceptable salt thereof.

The term “SGLT-2 inhibitor” refers to inhibitors of the sodium glucose cotransporter 2 such as especially atigliflozin, bexagliflozin, canagliflozin, dapagliflozin, empagliflozin, ertugliflozin, henagliflozin, ipragliflozin, luseogliflozin, remogliflozin, sotagliflozin, tianagliflozin, or tofogliflozin (especially canagliflozin, or dapagliflozin, or empagliflozin).

The term “DPP-4 inhibitor” or “DPP-IV inhibitor” refers to inhibitors of dipeptidyl peptidase 4 such as especially sitagliptin, vildagliptin, saxagliptin, and linagliptin, as well as gemigliptin, anagliptin, teneligliptin, alogliptin, trelagliptin, omarigliptin, evogliptin, and dutogliptin.

The term “GLP-1 receptor agonist” refers to agonists of the glucagon-like peptide-1 receptor such as especially exenatide, liraglutide, lixisenatide, albiglutide, dulaglutide, taspoglutide, semaglutide.

The term “dual GLP-1/GIP receptor agonist” refers to dual agonists of the glucagon-like peptide-1 receptor and the glucose-dependent insulinotropic polypeptide (GIP) receptor, such as especially trizepatide.

The term “sulfonylurea” refers especially to glibenclamide (glyburide), glibomuride, gliclazide, glipizide, gliquidone, glisoxepide, glyclopyramide, or glimepiride.

The term “thiazolidinediones” abbreviated as TZD, also known as glitazones, refers to agonists of the PPARγ (peroxisome proliferator-activated receptor gamma), and refers especially to pioglitazone, rosiglitazone, or lobeglitazone.

The term “diuretic” in the present application refers to loop diuretics including furosemide, bumetanide, ethacrynic acid, torsemide; potassium-sparing diuretics including for example amiloride, and notably including aldosterone antagonists (or alternatively named: mineralocorticoid receptor antagonists (MRA)) such as spironolactone, eplerenone, or finerenone; or aldosterone synthase inhibitors (such as baxdrostat); carbonic anhydrase inhibitors including acetazolamide and methazolamide; and in particular to diuretics of the thiazide class (thiazide-like diuretics) such as especially chlorthalidone, hydrochlorothiazide, chlorothiazide, indapamide, or metolazone. Preferred thiazide-like diuretic are chlorthalidone or hydrochlorothiazide. For avoidance of doubt, even though having a diuretic pharmacological effect, SGLT-2 inhibitors are not encompassed in the term “diuretic” as used herein. Furthermore, certain potassium-sparing diuretics may be considered predominantly for their pharmacological action as mineralocorticoid receptor antagonists (MRA)/aldosterone antagonists (e.g. finerenone), or as aldosterone synthase inhibitors (e.g. baxdrostat), rather than for the diuretic action as potassium-sparing diuretic. Such potassium-sparing diuretics are included herein in the definition as diuretics. It is further understood that standard background therapy may comprise the combination of several diuretics as defined herein. Particular combinations of diuretics are (i) an aldosterone antagonist in combination with a thiazide-like diuretic; (ii) an aldosterone antagonist in combination with a loop diuretic; (iii) an aldosterone synthase inhibitor in combination with a thiazide-like diuretic; (iv) an aldosterone synthase inhibitor in combination with a loop diuretic; and (v) a loop diuretic in combination with a thiazide-like diuretic.

The term “beta blocker” refers to such beta-adrenergic blocking agent, especially to acebutolol, atenolol, bisoprolol, metoprolol (including immediate release or sustained release formulations), nadolol, nebivolol, and propranolol (including immediate release or sustained release formulations); in particular to atenolol, bisoprolol, metoprolol, nebivolol, and propranolol.

Standard background therapy is preferably to be administered in a dosage corresponding to a tolerated efficacious dose of the respective active ingredient, e.g. when given as a single therapy. In particular, valsartan, or a pharmaceutically acceptable salt thereof, if present, is to be administered in a dosage form suitable for the oral administration of 160 mg or 320 mg per day of valsartan; losartan, or a pharmaceutically acceptable salt thereof, if present, is to be administered in a dosage form suitable for the oral administration of 50 mg or 100 mg per day of losartan; irbesartan, or a pharmaceutically acceptable salt thereof, if present, is to be administered in a dosage form suitable for the oral administration of 75 mg, 150 mg, or 300 mg per day of irbesartan; amlodipine, or a pharmaceutically acceptable salt thereof, if present, is to be administered in a dosage form suitable for the oral administration of 5 mg or 10 mg per day of amlodipine; enalapril, or a pharmaceutically acceptable salt thereof, if present, is to be administered in a dosage form suitable for the oral administration of 2.5 mg to 40 mg per day of enalapril; lisinopril, or a pharmaceutically acceptable salt thereof, if present, is to be administered in a dosage form suitable for the oral administration of 2.5 mg to 40 mg per day of lisinopril; ramipril, or a pharmaceutically acceptable salt thereof, if present, is to be administered in a dosage form suitable for the oral administration of 2.5 mg to 20 mg per day of ramipril; metformin, if present, is to be administered in a dosage form suitable for the oral administration of 500 mg to 2000 mg per day of metformin; glibenclamide if present, is to be administered in a dosage form suitable for the oral administration of 1.25 mg to 5 mg per day of glibenclamide; sitagliptin if present, is to be administered in a dosage form suitable for the oral administration of 25 mg to 100 mg per day of sitagliptin; vildagliptin if present, is to be administered in a dosage form suitable for the oral administration of two times 50 mg per day of vildagliptin; saxagliptin if present, is to be administered in a dosage form suitable for the oral administration of 2.5 mg or 5 mg per day of saxagliptin; linagliptin if present, is to be administered in a dosage form suitable for the oral administration of two times 5 mg per day of linagliptin; bexagliflozin, or a pharmaceutically acceptable salt thereof, if present, is to be administered in a dosage form suitable for the oral administration of 5 to 50 mg (in particular 20 mg) per day of bexagliflozin; canagliflozin, or a pharmaceutically acceptable salt thereof, if present, is to be administered in a dosage form suitable for the oral administration of of 50 to 400 mg (in particular 50 mg, 100 mg, 150 mg, or 300 mg; notably 100 mg, or 300 mg; especially 100 mg) per day of canagliflozin; dapagliflozin, or a pharmaceutically acceptable salt thereof, if present, is to be administered in a dosage form suitable for the oral administration of 1 to 20 mg (in particular 5 mg or 10 mg) per day of dapagliflozin; empagliflozin, or a pharmaceutically acceptable salt thereof, if present, is to be administered in a dosage form suitable for the oral administration of 5 to 50 mg (in particular 10 mg or 25 mg) per day of empagliflozin; ertugliflozin, or a pharmaceutically acceptable salt thereof, if present, is to be administered in a dosage form suitable for the oral administration of 2.5 to 50 mg (in particular 5 mg or 15 mg) per day of ertugliflozin; henagliflozin, or a pharmaceutically acceptable salt thereof, if present, is to be administered in a dosage form suitable for the oral administration of 5 to 100 mg (in particular 25 mg) per day of henagliflozin; ipragliflozin, or a pharmaceutically acceptable salt thereof, if present is to be administered in a dosage form suitable for the oral administration of 10 to 100 mg (in particular 25 mg or 50 mg) per day of ipragliflozin; luseogliflozin, or a pharmaceutically acceptable salt thereof, if present, is to be administered in a dosage form suitable for the oral administration of 1 to 10 mg (in particular 2.5 mg or 5 mg) per day of luseogliflozin; sotagliflozin, or a pharmaceutically acceptable salt thereof, if present, is to be administered in a dosage form suitable for the oral administration of 50 to 500 mg (in particular 75 mg, 200 mg or 400 mg) per day of sotagliflozin, tofogliflozin, or a pharmaceutically acceptable salt thereof, if present, is to be administered in a dosage form suitable for the oral administration of 10 to 50 mg (in particular 20 mg) per day of tofogliflozin.
10) A further aspect of the invention relates to the method according to any one of embodiments 1) to 9), wherein administering said clinically proven effective amount of aprocitentan results
   in a reduction of systolic blood pressure of at least about 12 mmHg (especially at least about 15 mmHg) from baseline after 4 weeks of treatment (wherein said reduction is measured by unattended Automated Office Blood Pressure Measurement (uAOBPM) at trough); and/or
   in a mean placebo corrected reduction of systolic blood pressure of at least 3.5 mmHg after 4 weeks of treatment (wherein said reduction is measured by unattended Automated Office Blood Pressure Measurement (uAOBPM) at trough).
11) A further embodiment relates to the method according to any one of embodiments 1) to 10), wherein administering said clinically proven effective amount of aprocitentan results in a reduction of diastolic blood pressure of at least about 8 mmHg (especially at least about 10 mmHg) from baseline after 4 weeks of treatment (wherein said reduction is measured by unattended Automated Office Blood Pressure Measurement (uAOBPM) at trough).
12) A further embodiment relates to the method according to any one of embodiments 1) to 11), wherein the administering said clinically proven effective amount of aprocitentan for an additional 32 weeks after said initial 4 weeks of treatment results at least in the maintenance of the mean reduction from baseline in systolic blood pressure and/or diastolic blood pressure for such additional 32 weeks.
13) A further embodiment relates to the method according any one of embodiments 1) to 9), wherein the clinical effect on blood pressure reduction (especially on systolic blood pressure reduction according to embodiment 10)) is confirmed after 4 weeks of a withdrawal period (starting 32 weeks after said initial 4 weeks of treatment), wherein the mean systolic blood pressure increases in subjects administered with placebo compared to the mean systolic blood pressure in subjects who continue to be administered with aprocitentan at a dose of 25 mg per day (i.e. by measuring the difference of mean SBP between week 36 and week 40 for placebo and aprocitentan, respectively, and comparing the mean SBPs); wherein said increase is at least about 5 mmHg, especially at least about 6 mmHg (wherein said increase is measured by unattended Automated Office Blood Pressure Measurement (uAOBPM) at trough).
14) A further embodiment relates to the method according to any one of embodiments 10) to 12), wherein said clinical effect on blood pressure reduction is confirmed by a mean reduction from baseline blood pressure after 4 weeks of treatment, wherein said reduction is measured by 24 hours ambulatory blood pressure monitoring (24 h ABPM); wherein especially
   systolic blood pressure is reduced by at least about 6 mmHg, and/or
      diastolic blood pressure is reduced by at least about 6 mmHg,
      wherein said clinically proven effective amount of aprocitentan is 12.5 mg per day; or
   systolic blood pressure is reduced by at least about 8 mmHg, and/or
      diastolic blood pressure is reduced by at least about 7 mmHg,
      wherein said clinically proven effective amount of aprocitentan is 25 mg per day.
15) A further embodiment relates to the method according to any one of embodiments 1) to 14), wherein the administering said clinically proven effective amount of aprocitentan results in a mean placebo corrected reduction of blood pressure after 4 weeks of treatment, wherein said reduction is measured by 24 hours ambulatory blood pressure monitoring (24 h ABPM) (wherein said BP is expressed as mean least squares value); wherein especially
   systolic blood pressure is reduced by at least about 4 mmHg, and/or
      diastolic blood pressure is reduced by at least about 5.5 mmHg,
      wherein said clinically proven effective amount of aprocitentan is 12.5 mg per day; or
   systolic blood pressure is reduced by at least about 4 mmHg, and/or
      diastolic blood pressure is reduced by at least about 5.5 mmHg,
      wherein said clinically proven effective amount of aprocitentan is 25 mg per day.
16) A further embodiment relates to the method according any one of embodiments 10) to 12), wherein said clinical effect on blood pressure reduction is confirmed by a mean reduction from baseline blood pressure after 4 weeks of treatment, wherein said reduction is measured by nighttime ambulatory blood pressure monitoring (nighttime ABPM); wherein especially
   systolic blood pressure is reduced by at least about 6 mmHg, especially at least about 8 mmHg, and/or
      diastolic blood pressure is reduced by at least about 6 mmHg, especially at least about 7 mmHg,
      wherein said clinically proven effective amount of aprocitentan is 12.5 mg per day; or
   systolic blood pressure is reduced by at least about 8 mmHg, especially at least about 10 mmHg, and/or
      diastolic blood pressure is reduced by at least about 7 mmHg, especially at least about 8 mmHg,
      wherein said clinically proven effective amount of aprocitentan is 25 mg per day.
17) A further embodiment relates to the method according to any one of embodiments 1) to 16), wherein the administering said clinically proven effective amount of aprocitentan results in a mean placebo corrected reduction of blood pressure after 4 weeks of treatment, wherein said reduction is measured by nighttime ambulatory blood pressure monitoring (nighttime ABPM) (wherein said BP is expressed as mean least squares value); wherein especially
   systolic blood pressure is reduced by at least about 4 mmHg, especially at least about 5 mmHg, and/or
      diastolic blood pressure is reduced by at least about 4 mmHg, especially at least about 5 mmHg,
      wherein said clinically proven effective amount of aprocitentan is 12.5 mg per day; or
   systolic blood pressure is reduced by at least about 4 mmHg, especially at least about 7 mmHg, and/or
      diastolic blood pressure is reduced by at least about 5.5 mmHg, especially at least about 6 mmHg,
      wherein said clinically proven effective amount of aprocitentan is 25 mg per day.
18) A further embodiment relates to the method according any one of embodiments 1) to 9), wherein the reduction of blood pressure (e.g. as defined in embodiment 10) or 11) is confirmed after 4 weeks of a withdrawal period (starting 32 weeks after said initial 4 weeks of treatment), wherein the mean blood pressure increases in subjects administered with placebo compared to the respective mean blood pressure in subjects who continue to be administered with aprocitentan at a dose of 25 mg per day (i.e. by measuring the difference of the respective mean BP between week 36 and week 40 for placebo and aprocitentan, respectively, and comparing the respective mean BPs); wherein said increase is measured by 24 hours ambulatory blood pressure monitoring (24h ABPM) (wherein said BP is expressed as mean least squares value); wherein
   systolic blood pressure is increased by at least about 6 mmHg, especially at least about 6.5 mmHg, and/or
      diastolic blood pressure is increased by at least about 6 mmHg, especially at least about 6.5 mmHg.
19) A further embodiment relates to the method according any one of embodiments 1) to 9), wherein the reduction of blood pressure (e.g. as defined in embodiment 10) or 11)) is confirmed after 4 weeks of a withdrawal period (starting 32 weeks after said initial 4 weeks of treatment), wherein the mean blood pressure increases in subjects administered with placebo compared to the respective mean blood pressure in subjects who continue to be administered with aprocitentan at a dose of 25 mg per day (i.e. by measuring the difference of the respective mean BP between week 36 and week 40 for placebo and aprocitentan, respectively, and comparing the respective mean BPs); wherein said increase is measured by nighttime ambulatory blood pressure monitoring (nighttime ABPM) (wherein said BP is expressed as mean least squares value); wherein
   systolic blood pressure is increased by at least about 8 mmHg, especially at least about 8.5 mmHg, and/or
      diastolic blood pressure is increased by at least about 7 mmHg, especially at least about 7.5 mmHg.
20) A further aspect of the invention relates to the method according to any one of embodiments 1) to 19), wherein said method of treating hypertension including resistant hypertension in a subject in need thereof comprises administering to the subject a pharmaceutical composition comprising a clinically proven safe and clinically proven effective amount of aprocitentan.
21) A further embodiment relates to the method according to any one of embodiments 1) to 20), wherein the clinically proven effective amount (and, respectively, clinically proven safe amount according to embodiment
20) is 10 to 50 mg; especially 10 mg, 12.5 mg, 20 mg, 25 mg, 30 mg, 40 mg, or 50 mg; in particular 12.5 mg or 25 mg; per day of aprocitentan.
22) A further embodiment relates to the method according to any one of embodiments 1) to 20), wherein the clinically proven effective amount (and, respectively, clinically proven safe amount according to embodiment 20) is 12.5 mg per day of aprocitentan.
23) A further embodiment relates to the method according to any one of embodiments 1) to 20), wherein the clinically proven effective amount (and, respectively, clinically proven safe amount according to embodiment 20) is 25 mg per day of aprocitentan.
23) A further embodiment relates to the method according to any one of embodiments 21) to 23), wherein, mutatis mutandis, said pharmaceutical composition comprises aprocitentan, or a pharmaceutically acceptable salt thereof, in a pharmaceutical unit dosage form suitable for the oral administration of 10 to 50 mg; especially 10 mg, 12.5 mg, 20 mg, 25 mg, 30 mg, 40 mg, or 50 mg; in particular 12.5 mg or 25 mg; per day of aprocitentan.
24) A further embodiment relates to the method according to any one of embodiments 1) to 23), wherein said pharmaceutical composition comprising aprocitentan, or a pharmaceutically acceptable salt thereof, is administered in the morning (wherein it is understood that aprocitentan is suitable for administration once a day).

Unless used regarding temperatures, the term “about” placed before a numerical value “X” refers in the current application to an interval extending from X minus 10% of X to X plus 10% of X, and preferably to an interval extending from X minus 5% of X to X plus 5% of X. In the particular case of temperatures, the term “about” placed before a temperature “Y” refers in the current application to an interval extending from the temperature Y minus 10° C. to Y plus 10° C., preferably to an interval extending from Y minus 5° C. to Y plus 5° C., notably to an interval extending from Y minus 3° C. to Y plus 3° C. Room temperature means a temperature of about 25° C.

When in the current application the term n equivalent(s) is used wherein n is a number, it is meant and within the scope of the current application that n is referring to about the number n, preferably n is referring to the exact number n.

Whenever the word “between” or “to” is used to describe a numerical range, it is to be understood that the end points of the indicated range are explicitly included in the range. For example: if a temperature range is described to be between 40° C. and 80° C. (or 40° C. to 80° C.), this means that the end points 40° C. and 80° C. are included in the range; or if a variable is defined as being an integer between 1 and 4 (or 1 to 4), this means that the variable is the integer 1, 2, 3, or 4.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the crystalline forms of the present invention, optionally in combination with other therapeutically valuable substances, into a galenical administration form together with suitable, non-toxic, inert, pharmaceutically acceptable solid or liquid carrier materials and, if desired, usual pharmaceutical adjuvants.

An example of a solid pharmaceutical composition (in particular in the form of a tablet) comprises as pharmaceutically acceptable excipient inert microcrystalline cellulose, lactose, hydroxypropylcellulose, croscarmellose sodium and magnesium stearate.

Especially, such solid pharmaceutical composition will comprise aprocitentan in a total amount from 5 to 25% in weight based on the total weight of the pharmaceutical composition, microcrystalline cellulose in a total amount from 20 to 30% in weight based on the total weight of the pharmaceutical composition, lactose in a total amount from 40 to 65% in weight based on the total weight of the pharmaceutical composition, hydroxypropylcellulose in a total amount from 1 to 3% in weight based on the total weight of the pharmaceutical composition, croscarmellose sodium in a total amount from 2 to 8% in weight based on the total weight of the pharmaceutical composition and magnesium stearate in a total amount from 0.2 to 2% in weight based on the total weight of the pharmaceutical composition, whereby the total percent in weight of the solid pharmaceutical composition will always be 100; the aforementioned solid pharmaceutical composition will particularly be in the form of a tablet.

A further embodiment of the invention relates to such pharmaceutical composition, wherein said pharmaceutical composition is in form of a tablet. In a sub-embodiment, the pharmaceutically active ingredients are comprised in granules prior to compression to said tablet. A tablet can optionally be coated with a suitable protective pellicle. Said protective pellicle will notably prevent direct contact of the pharmaceutical composition with moisture; it may also ease imprints that may be desired to be used in order to distinguish the pharmaceutical composition from others. The coating material for making such protective pellicle may include a low water vapour permeability polymer (such as a polyvinyl alcohol (e.g. Aquapolish® white PVA from manufacturer Biogrund) or dimethylaminoethyl methacrylate (e.g. EUDRAGIT® E PO)). The coating material can further include a plasticizing agent (e.g. propylene glycol, triacetyne, dibutyl phthalate or dibutyl sebacate), a surfactant (e.g. sodium lauryl sulphate or a polysorbate such as Tween®) and/or a lubricant/glidant (e.g. stearic acid, magnesium or calcium stearate or talc). Moreover, the coating material can also include a pigment (e.g. iron (II) oxide, iron (III) oxide or titanium oxide) to give the tablet a coloured aspect.

In the context of the present invention, endothelin related diseases include
hypertension including essential hypertension, and especially difficult to control and resistant hypertension;
pulmonary hypertension and pulmonary arterial hypertension;
chronic kidney disease (CKD) [especially CKD of stages 1 to 4 as defined by the Kidney Disease Improving Global Outcomes (KDIGO) Guidelines (and notably CKD of stage 3 or 4)], and in particular CKD (notably of these stages) caused by/associated with hypertension, and/or caused by/associated with diabetes (diabetic kidney disease (DKD)); as well as (acute and, especially, chronic) renal failure wherein said (acute and chronic) renal failure may be associated with hypertension and/or diabetes; diabetic nephropathy wherein said diabetic nephropathy may be associated with hypertension; and glomerulonephritis wherein said glomerulonephritis may be associated with hypertension and/or diabetes;
   For avoidance of doubt, the term CKD caused by/associated with diabetes (diabetic kidney disease, DKD) may also include such DKD associated, in addition, with hypertension; wherein especially the diabetes is type 2 diabetes.
   in a sub-embodiment, DKD as defined before especially refers to DKD in a patient diagnosed with type 2 diabetes mellitus; in particular to the reduction of the rate of progression of DKD e.g. in a patient diagnosed with type 2 diabetes mellitus, wherein such reduced rate of progression may especially be expressed by a reduction in eGFR, a reduction of events of end-stage kidney disease (ESKD), or a reduction of events of renal death; wherein especially said patient presents in addition a history of hypertension;
   in a further sub-embodimment, DKD as defined before especially refers to diabetic nephropathy associated with an elevated serum creatinine and/or proteinuria [especially corresponding to CKD of stages 1 to 4 as defined by the Kidney Disease Improving Global Outcomes (KDIGO) Guidelines (and notably to CKD of stage 3 or 4)] in patients with type 2 diabetes, especially in such patients presenting in addition a history of hypertension;
   in a further sub-embodimment, DKD as defined before especially refers to such DKD associated, in addition, with hypertension; wherein especially the diabetes is type 2 diabetes;
diabetes, and diabetes related diseases such as diabetic arteriopathy, diabetic retinopathy, or diabetic vasculopathy; as well as therapy and prophylaxis of diabetic complications; and reducing the risk of developing a major cardiovascular event (such as HF, myocardial infarction, stroke, or death from cardiovascular causes) in patients who have diabetes, especially in patients who have diabetes that is accompanied by at least one other cardiovascular risk factor (such as especially hypertension); as well as diabetic foot ulcers and/or reducing the risk of lower extremety amputations in patients who have diabetes; and
heart failure (HF) defined as including especially chronic HF, including in particular systolic HF/HF with reduced ejection fraction (HFrEF) (i.e. ejection fraction<about 40%), and diastolic HF/HF with preserved ejection fraction (HFpEF) (i.e. ejection fraction>about 50%); as well as reducing the risk of developing a major cardiovascular event (such as HF, myocardial infarction, stroke, or death from cardiovascular causes) in patients who are at cardiovascular risk (such as patients who have coronary artery disease and/or patients who have demonstrated clinical signs of congestive HF); angina pectoris; coronary diseases; cardiac insufficiency; and diastolic dysfunction.

Notably, in the context of the present invention, endothelin related diseases refer to hypertension, especially difficult to control or resistant hypertension.

Especially, in the context of the present invention, endothelin related diseases refer to resistant hypertension.

Essential hypertension (also called primary hypertension or idiopathic hypertension) is the form of hypertension that by definition has no identifiable cause. It represents a significant global public health concern, contributing to vascular and renal morbidity and to cardiovascular mortality. The diagnosis of essential hypertension is made when the average of multiple systolic blood pressure measurements on 2 or more subsequent visits is consistently equal to or above a certain threshold value TsBp. Individuals with high normal blood pressure tend to maintain pressures that are above average for the general population and are at greater risk for development of definite hypertension and cardiovascular events than the general population. The threshold value T_{SBP} above which treatment is recommended is regularly discussed among clinicians (see e.g. Mancia et al, J. Hypertens. (2013), 31, 1281-1357); accordingly, depending on the patient's general condition and age, T_{SBP} could be 140 or 130 mm Hg, or another suitable value.

The term “difficult to control hypertension” in the present invention is defined as blood pressure that remains above goal in spite of the concurrent use of standard background therapy, wherein said standard background therapy comprises treatment with at least one, especially with at least two, in particular at least three (in which case the term is used equivalent to resistant hypertension as defined below) antihypertensive agents of different classes. Where said standard background therapy comprises more than one therapeutic agents, one of said therapeutic agents should be a diuretic and all agents should be prescribed at optimal/maximal dose amounts.

The term “resistant hypertension” in the present invention is defined as blood pressure that remains above goal in spite of the concurrent use of three antihypertensive agents of different classes. One of the three therapeutic agents should be a diuretic and all agents should be prescribed at optimal/maximal dose amounts. As defined, resistant hypertension patients include patients whose blood pressure is controlled with use of more than three medications. That is, patients whose blood pressure is controlled but require four or more medications to do so should be considered resistant to treatment (see e.g. Mancia et al, J. Hypertens. (2013), 31, 1281-1357).

The term “diabetes” as used herein refers to all types of diabetes, especially to type 2 diabetes; as well as type 1 diabetes and latent autoimmune diabetes of adulthood, a form of diabetes mellitus type 1 that occurs in adulthood, often with a slower course of onset than type 1 diabetes diagnosed in juveniles.
25) A further aspect of the invention relates, mutatis mutandis, to the method according to any one of embodiments 1) to 24), wherein said method is for.
   the treatment of CKD [especially CKD of stages 1 to 4, notably CKD of stage 3 or 4]; and in particular CKD (notably of these stages) caused by/associated with hypertension, and/or caused by/associated with diabetes (DKD); as well as in the prophylaxis/prevention or treatment of acute or chronic renal failure; diabetic nephropathy; or glomerulonephritis;
      wherein, in a first sub-embodiment, such use is especially for the treatment of such DKD in a patient diagnosed with type 2 diabetes mellitus, wherein in particular aprocitentan reduces the rate of progression of DKD, wherein such reduced rate of progression may especially be expressed by a reduction in eGFR, a reduction of events of end-stage kidney disease (ESKD), or a reduction of events of renal death; wherein notably said patient presents in addition a history of hypertension;
      wherein, in a second sub-embodimment, such use is especially for the treatment of such DKD, including treatment of diabetic nephropathy associated with an elevated serum creatinine and/or proteinuria [especially corresponding to CKD of stages 1 to 4 as defined by the Kidney Disease Improving Global Outcomes (KDIGO) Guidelines (and notably to such CKD of stage 3 or 4)], in patients with type 2 diabetes, especially in such patients presenting in addition a history of hypertension;
      wherein in a variant, said method according to any one of embodiments 1) to 24) is for the treatment of hypertension in a subject who is diagnosed with CKD [especially CKD of stages 1 to 4, notably CKD of stage 3 or 4]; and in particular diagnosed with CKD [especially CKD of stages 1 to 4, notably CKD of stage 3 or 4] and diabetes (especially diabetes of type 2) (DKD); wherein notably, in addition to reducing blood pressure according to any one of embodiments 10) to 19), said clinically proven effective amount of aprocitentan, or a pharmaceutically acceptable salt thereof; reduces the rate of progression of CKD, respectively DKD, wherein such reduced rate of progression may especially be expressed by a reduction of urinary albumin-creatinine ratio (UACR), a maintenance of eGFR/a prevention of further decline in eGFR, a reduction of events of end-stage kidney disease (ESKD), and/or a reduction of events of renal death;
   the treatment of diabetes, and diabetes related diseases such as diabetic arteriopathy, diabetic retinopathy, or diabetic vasculopathy; as well as diabetic complications; for reducing the risk of developing a major cardiovascular event (such as HF, myocardial infarction, stroke, or death from cardiovascular causes) in patients who have diabetes, especially in patients who have diabetes that is accompanied by at least one other cardiovascular risk factor (such as especially hypertension); as well as for use in the prophylaxis/prevention or treatment of diabetic foot ulcers and/or for reducing the risk of lower extremety amputations in patients who have diabetes;
   the treatment of heart failure (HF) including especially chronic HF, including in particular systolic HF and diastolic HF; for reducing the risk of developing a major cardiovascular event (such as HF, myocardial infarction, stroke, or death from cardiovascular causes) in patients who are at cardiovascular risk (such as patients who have coronary artery disease and/or patients who have demonstrated clinical signs of congestive HF); as well as for use in the prophylaxis/prevention or treatment of ischemic heart diseases including angina pectoris, coronary diseases, and myocardial ischemia; cardiac insufficiency; or diastolic dysfunction;
   the treatment of atherosclerosis; as well as of peripheral arterial obliterant disease including chronic peripheral arteriopathy;
   the treatment of digital ulcers; or
   the treatment of connective tissue diseases.
26) A further aspect of the invention relates, mutatis mutandis, to the method according to any one of embodiments 1) to 24), wherein said method is for
   for the treatment of CKD [especially CKD of stages 1 to 4, notably CKD of stage 3 or 4] caused by/associated with hypertension; and/or
   for the treatment of CKD [especially CKD of stages 1 to 4, notably CKD of stage 3 or 4] caused by/associated with diabetes (DKD);
      wherein such use is especially for the treatment of such DKD in a patient diagnosed with type 2 diabetes mellitus, wherein in particular aprocitentan reduces the rate of progression of DKD, wherein such reduced rate of progression may especially be expressed by a reduction in eGFR, a reduction of events of end-stage kidney disease (ESKD), or a reduction of events of renal death; wherein notably said patient presents in addition a history of hypertension;
      wherein in a variant, said method according to any one of embodiments 1) to 24) is for the treatment of hypertension in a subject who is diagnosed with CKD [especially CKD of stages 1 to 4, notably CKD of stage 3 or 4]; and in particular diagnosed with CKD [especially CKD of stages 1 to 4, notably CKD of stage 3 or 4] and diabetes (especially diabetes of type 2) (DKD); wherein notably, in addition to reducing blood pressure according to any one of embodiments 10) to 19), said clinically proven effective amount of aprocitentan, or a pharmaceutically acceptable salt thereof; reduces the rate of progression of CKD, respectively DKD, wherein such reduced rate of progression may especially be expressed by a reduction of urinary albumin-creatinine ratio (UACR), a maintenance of eGFR/a prevention of further decline in eGFR, a reduction of events of end-stage kidney disease (ESKD), and/or a reduction of events of renal death; and/or
   reducing the risk of developing a major cardiovascular event (such as HF, myocardial infarction, stroke, or death from cardiovascular causes) in patients who have diabetes, especially in patients who have diabetes that is accompanied by at least one other cardiovascular risk factor (such as especially hypertension).
27) A further aspect of the invention relates, mutatis mutandis, to the method according to any one of embodiments 1) to 24), wherein said method is for
   for treatment of CKD [especially CKD of stages 1 to 4, notably CKD of stage 3 or 4] caused by/associated with hypertension; and/or
   for treatment of CKD [especially CKD of stages 1 to 4, notably CKD of stage 3 or 4] caused by/associated with diabetes (DKD);
      wherein such use is especially for the treatment of such DKD in a patient diagnosed with type 2 diabetes mellitus, wherein in particular aprocitentan reduces the rate of progression of DKD, wherein such reduced rate of progression may especially be expressed by a reduction in eGFR, a reduction of events of end-stage kidney disease (ESKD), or a reduction of events of renal death; wherein notably said patient presents in addition a history of hypertension;
      wherein in a variant, said method according to any one of embodiments 1) to 24) is for the treatment of hypertension in a subject who is diagnosed with CKD [especially CKD of stages 1 to 4, notably CKD of stage 3 or 4]; and in particular diagnosed with CKD [especially CKD of stages 1 to 4, notably CKD of stage 3 or 4] and diabetes (especially diabetes of type 2) (DKD); wherein notably, in addition to reducing blood pressure according to any one of embodiments 10) to 19), said clinically proven effective amount of aprocitentan, or a pharmaceutically acceptable salt thereof; reduces the rate of progression of CKD, respectively DKD, wherein such reduced rate of progression may especially be expressed by a reduction of urinary albumin-creatinine ratio (UACR), a maintenance of eGFR/a prevention of further decline in eGFR, a reduction of events of end-stage kidney disease (ESKD), and/or a reduction of events of renal death.
27) A further aspect of the invention relates, mutatis mutandis, to the method according to any one of embodiments 1) to 24), wherein said method is for
   for the treatment of CKD [especially CKD of stages 1 to 4, notably CKD of stage 3 or 4] caused by/associated with diabetes (DKD);
      wherein, in a first sub-embodiment, such use is especially for the treatment of such DKD in a patient diagnosed with type 2 diabetes mellitus, wherein in particular aprocitentan reduces the rate of progression of DKD, wherein such reduced rate of progression may especially be expressed by a reduction in eGFR, a reduction of events of end-stage kidney disease (ESKD), or a reduction of events of renal death; wherein notably said patient presents in addition a history of hypertension;
      wherein, in a second sub-embodimment, such use is especially for the treatment of such DKD, including treatment of diabetic nephropathy associated with an elevated serum creatinine and/or proteinuria [especially corresponding to CKD of stages 1 to 4 as defined by the Kidney Disease Improving Global Outcomes (KDIGO) Guidelines (and notably to such CKD of stage 3 or 4)], in patients with type 2 diabetes, especially in such patients presenting in addition a history of hypertension;
      wherein in a variant, said method according to any one of embodiments 1) to 24) is for the treatment of hypertension in a subject who is diagnosed with CKD [especially CKD of stages 1 to 4, notably CKD of stage 3 or 4]; and in particular diagnosed with CKD [especially CKD of stages 1 to 4, notably CKD of stage 3 or 4] and diabetes (especially diabetes of type 2) (DKD); wherein notably, in addition to reducing blood pressure according to any one of embodiments 10) to 19), said clinically proven effective amount of aprocitentan, or a pharmaceutically acceptable salt thereof; reduces the rate of progression of CKD, respectively DKD, wherein such reduced rate of progression may especially be expressed by a reduction of urinary albumin-creatinine ratio (UACR), a maintenance of eGFR/a prevention of further decline in eGFR, a reduction of events of end-stage kidney disease (ESKD), and/or a reduction of events of renal death.
28) A further aspect of the invention relates, mutatis mutandis, to the method according to any one of embodiments 1) to 24), wherein said method is for
   reducing the risk of developing a major cardiovascular event (such as HF, myocardial infarction, stroke, or death from cardiovascular causes) in patients who have diabetes, especially in patients who have diabetes that is accompanied by at least one other cardiovascular risk factor (such as especially hypertension).

As used herein, unless otherwise noted, the term “clinically proven” (used independently or to modify the terms “safe” and/or “effective”) shall mean that it has been proven by a clinical trial wherein the clinical trial has met the approval standards of U.S. Food and Drug Administration, EMA or a corresponding national regulatory agency. For example, the clinical study may be an adequately sized, randomized, double-blinded study used to clinically prove the effects of the drug.

The terms “clinically proven efficacy” and “clinically proven effective” as used herein in the context of a dose, dosage regimen, treatment or method refer to the effectiveness of a particular dose, dosage or treatment regimen. Efficacy can be measured based on change in the course of the disease in response to an agent of the present invention. For example, a dual endothelian receptor antagonist of the present invention (especially aprocitentan) is administered to a subject in an amount and for a time sufficient to induce an improvement, preferably a sustained improvement, in at least one indicator that reflects the severity of the disorder that is being treated. Various indicators that reflect the extent of the subject's illness, disease or condition can be assessed for determining whether the amount and time of the treatment is sufficient. Such indicators include, for example, clinically recognized indicators of disease severity, symptoms, or manifestations of the disorder in question. The degree of improvement generally is determined by a physician, who can make this determination based on signs, symptoms, biopsies, or other test results, and who can also employ questionnaires that are administered to the subject, such as quality-of-life questionnaires developed for a given disease. For example, a compound of the present invention can be administered to achieve an improvement in a subject's condition related to hypertension including resistant hypertension. Improvement can be indicated by an improvement in an index of disease activity, by amelioration of clinical symptoms or by any other measure of disease activity.

In a particular embodiment, such index of disease is the blood pressure including systolic and diastolic blood pressure or reduction thereof as measured via uAOBPM and/or ABPM (including 24 h ABPM and nighttime ABPM), each as compared to baseline or as compared to placebo; e.g. according to any one of embodiments 10) to 19).

In certain embodiments, a clinically proven safe amount of aprocitentan is an amount that results in a reduction in blood pressure [for example as measured via uAOBPM and/or ABPM (including 24 h ABPM and nighttime ABPM), each as compared to baseline or as compared to placebo; e.g. according to any one of embodiments 10) to 19)]; as demonstrated via clinical trial having the protocol as described in Example A or herein below.

Nocturnal hypertension can be defined, according to K. Kario, Hypertension. 2018;71:997-1009, as night-time BP≥120/70 mm Hg (>110/65 mm Hg by the new 2017 ACC/AHA guidelines). Clinic and morning home BP of <130/80 mm Hg is defined as masked nocturnal hypertension and as masked uncontrolled nocturnal hypertension under a medicated condition. The night-time BP is calculated as the average of night-time BPs (from going to bed to arising) measured by ABPM or by the recently developed HBPM system with an automated measurement function for night-time BP (night-time HBPM). The number of night-time BP measurements required may be ≥6. The night-time systolic BP dipping (%) is calculated as (1-average night-time systolic BP/average daytime systolic BP)×100, and based on this percentage, the following 4 night-time BP dipping patterns are defined: extreme dipper: >20%; dipper: ≤20%->10%; nondipper: ≤10%->0%; riser: ≤0%. This classification is usually based on the ABPM data.
29) A further embodiment of the invention, thus, relates to a method of treating endothelin related disorders comprising especially hypertension including difficult to control and resistant hypertension; chronic kidney disease (CKD) [notably CKD of stages 1 to 4 as defined by the Kidney Disease Improving Global Outcomes (KDIGO) Guidelines (and especially CKD of stage 3 or 4)], and in particular CKD (notably of these stages) caused by/associated with hypertension and/or caused by/associated with diabetes (diabetic kidney disease (DKD) according to any one of embodiments 1) to 24); said endothelin related disorders comprising especially hypertension including difficult to control and resistant hypertension; said method comprising administering to the subject a pharmaceutical composition comprising a clinically proven safe and clinically proven effective amount of aprocitentan, or a pharmaceutically acceptable salt thereof; wherein said clinically proven safe and clinically proven effective amount of aprocitentan is an amount that results in a reduction in blood pressure; wherein said clinically proven safe and clinically proven effective amount of aprocitentan results in a mean placebo corrected reduction of blood pressure after 4 weeks of treatment, wherein said reduction is measured by 24 hours ambulatory blood pressure monitoring (24 h ABPM) (wherein said BP is expressed as mean least squares value) according to embodiment 15), and/or said clinically proven safe and clinically proven effective amount of aprocitentan results in a mean placebo corrected reduction of blood pressure after 4 weeks of treatment, wherein said reduction is measured by nighttime ambulatory blood pressure monitoring (nighttime ABPM) (wherein said BP is expressed as mean least squares value) according to embodiment 17); wherein especially the subject treated shows nocturnal hypertension, notably nocturnal hypertension associated with a night-time systolic BP dipping pattern that can be defined as dipper, nondipper or riser; especially as nondipper or riser as measured by ABPM; wherein especially said subject is diagnosed with CKD [especially CKD of stages 1 to 4, notably CKD of stage 3 or 4]; and in particular said subject is diagnosed with CKD [especially CKD of stages 1 to 4, notably CKD of stage 3 or 4] associated with diabetes (especially diabetes of type 2) (DKD); wherein said clinically proven safe and clinically proven effective amount reduces, in addition, the rate of progression of CKD, respectively DKD, wherein such reduced rate of progression may especially be expressed by a reduction of urinary albumin-creatinine ratio (UACR), a maintenance of eGFR/a prevention of further decline in eGFR, a reduction of events of end-stage kidney disease (ESKD), and/or a reduction of events of renal death; wherein said pharmaceutical composition comprises aprocitentan, or a pharmaceutically acceptable salt thereof, in a pharmaceutical unit dosage form suitable for the oral administration of 12.5 mg or 25 mg (in particular 25 mg); per day of aprocitentan.
30) A further embodiment of the invention relates to a method of treating endothelin related disorders according to any one of embodiments 1) to 24); comprising especially hypertension including difficult to control and resistant hypertension, in a subject in need thereof, comprising administering to the subject a pharmaceutical composition comprising a clinically proven safe and clinically proven effective amount of aprocitentan, or a pharmaceutically acceptable salt thereof; wherein said clinically proven safe and clinically proven effective amount of aprocitentan is an amount that results in a reduction in blood pressure as measured via nighttime ABPM (as compared to baseline or as compared to placebo; e.g. according to any one of embodiments 10) to 19), for example as demonstrated via clinical trial having the protocol as described in Example A or herein below); wherein the subject treated shows nocturnal hypertension, notably nocturnal hypertension associated with a night-time systolic BP dipping pattern that can be defined as dipper, nondipper or riser; especially as nondipper or riser as measured by ABPM; wherein especially said subject is diagnosed with CKD [especially CKD of stages 1 to 4, notably CKD of stage 3 or 4]; and in particular said subject is diagnosed with CKD [especially CKD of stages 1 to 4, notably CKD of stage 3 or 4] associated with diabetes (especially diabetes of type 2) (DKD); wherein said clinically proven safe and clinically proven effective amount reduces, in addition, the rate of progression of CKD, respectively DKD, wherein such reduced rate of progression may especially be expressed by a reduction of urinary albumin-creatinine ratio (UACR), a maintenance of eGFR/a prevention of further decline in eGFR, a reduction of events of end-stage kidney disease (ESKD), and/or a reduction of events of renal death; wherein said pharmaceutical composition comprises aprocitentan, or a pharmaceutically acceptable salt thereof, in a pharmaceutical unit dosage form suitable for the oral administration of 12.5 mg or 25 mg (in particular 25 mg); per day of aprocitentan.
31) A further embodiment relates to the method according to any one of embodiments 1) to 30), said method comprising administering to the subject a pharmaceutical composition comprising a clinically proven safe and clinically proven effective amount of aprocitentan, or a pharmaceutically acceptable salt thereof; wherein the administering said clinically proven safe and clinically proven effective amount of aprocitentan results in a mean placebo corrected reduction of blood pressure after 4 weeks of treatment, wherein said reduction is measured by 24 hours ambulatory blood pressure monitoring (24 h ABPM) (wherein said BP is expressed as mean least squares value); wherein especially.
   systolic blood pressure is reduced by at least about 4 mmHg, and/or
      diastolic blood pressure is reduced by at least about 5.5 mmHg,
      wherein said clinically proven safe and clinically proven effective amount of aprocitentan is 12.5 mg per day; or
   systolic blood pressure is reduced by at least about 4 mmHg, and/or
      diastolic blood pressure is reduced by at least about 5.5 mmHg,
      wherein said clinically proven safe and clinically proven effective amount of aprocitentan is 25 mg per day;

and wherein, in each case, preferably said subject has CKD of stage 3 or 4 (optionally associated with diabetes mellitus), wherein the mean placebo corrected UACR after 4 weeks of treatment is reduced by at least about 25% (especially by at least about 30%);

and wherein notably, in case said subject has macroproteinuria (defined as UACR >300 mg/g),
the mean placebo corrected UACR after 4 weeks of treatment is reduced by at least about 40% (especially about 45%), wherein said clinically proven safe and clinically proven effective amount of aprocitentan is 12.5 mg per day; or.
the mean placebo corrected UACR after 4 weeks of treatment is reduced by at least about 50% (especially about 60%), wherein said clinically proven safe and clinically proven effective amount of aprocitentan is 25 mg per day;

and wherein preferably said clinically proven safe and clinically proven effective amount of aprocitentan is 25 mg per day (wherein preferably said amount is (to be) administered once a day in the morning).
32) A further embodiment relates to the method according to any one of embodiments 1) to 30), said method comprising administering to the subject a pharmaceutical composition comprising a clinically proven safe and clinically proven effective amount of aprocitentan, or a pharmaceutically acceptable salt thereof; wherein the administering said clinically proven safe and clinically proven effective amount of aprocitentan results in a mean placebo corrected reduction of blood pressure after 4 weeks of treatment, wherein said reduction is measured by nighttime ambulatory blood pressure monitoring (nighttime ABPM) (wherein said BP is expressed as mean least squares value); wherein especially.
   systolic blood pressure is reduced by at least about 4 mmHg, especially at least about 5 mmHg, and/or diastolic blood pressure is reduced by at least about 4 mmHg, especially at least about 5 mmHg, wherein said clinically proven safe and clinically proven effective amount of aprocitentan is 12.5 mg per day; or
   systolic blood pressure is reduced by at least about 4 mmHg, especially at least about 7 mmHg, and/or diastolic blood pressure is reduced by at least about 5.5 mmHg, especially at least about 6 mmHg, wherein said clinically proven safe and clinically proven effective amount of aprocitentan is 25 mg per day;

and wherein, in each case, preferably said subject has CKD of stage 3 or 4 (optionally associated with diabetes mellitus), wherein the mean placebo corrected UACR after 4 weeks of treatment is reduced by at least about 25% (especially by at least about 30%);

and wherein notably in case said subject has macroproteinuria (defined as UACR>300 mg/g, e.g. as measured at baseline),
the mean placebo corrected UACR after 4 weeks of treatment is reduced by at least about 40% (especially about 45%), wherein said clinically proven safe and clinically proven effective amount of aprocitentan is 12.5 mg per day; or
   the mean placebo corrected UACR after 4 weeks of treatment is reduced by at least about 50% (especially about 60%), wherein said clinically proven safe and clinically proven effective amount of aprocitentan is 25 mg per day;

and wherein preferably said clinically proven safe and clinically proven effective amount of aprocitentan is 25 mg per day (wherein preferably said amount is (to be) administered once a day in the morning).

It is understood that, whenever a subject has/has a medical history of CKD and/or diabetes, for example a subject having been diagnosed as having DKD (i.e. CKD and diabetes), such subject may receive standard background therapy
to reduce the risk of cardiovascular death and hospitalization for heart failure in adults with heart failure, and/or
to reduce the risk of cardiovascular death in adults with type 2 diabetes mellitus and established cardiovascular disease; and/or
as an adjunct to diet and exercise to improve glycemic control in adults with type 2 diabetes mellitus; and/or
for the treatment of diabetes (in particular type 2 diabetes mellitus);

wherein especially such subject may receive (in addition to standard background therapy suitable for the treatment of hypertension) a standard background therapy suitable for the treatment of diabetes (in particular type 2 diabetes); wherein such standard background therapy in particular would comprise an SGLT-2 inhibitor (notably atigliflozin, bexagliflozin, canagliflozin, dapagliflozin, empagliflozin, ertugliflozin, henagliflozin, ipragliflozin, luseogliflozin, remogliflozin, sotagliflozin, or tofogliflozin; or a pharmaceutically acceptable salt thereof; especially bexagliflozin, canagliflozin, dapagliflozin, empagliflozin, ertugliflozin, henagliflozin, ipragliflozin, luseogliflozin, sotagliflozin, or tofogliflozin; in particular canagliflozin, or dapagliflozin, or empagliflozin), or a pharmaceutically acceptable salt thereof.

The term “clinically proven safe,” as it relates to a dose, dosage regimen, treatment or method with the compound of the present invention, aprocitentan, refers to a favorable risk: benefit ratio with an acceptable frequency and/or acceptable severity of treatment-emergent adverse events (referred to as AEs or TEAEs). As used herein, “adverse event,” “treatment-emergent adverse event,” and “adverse reaction” mean any harm, unfavorable, unintended or undesired sign or outcome associated with or caused by administration of a pharmaceutical composition or therapeutic. It is an untoward medical occurrence in a subject administered a medicinal product.

Accordingly, aprocitentan or a pharmaceutically acceptable salt thereof according to this invention comprises the use in combination (or co-therapy) with said further pharmaceutically active ingredients, including especially standard background therapy agents.

A combined treatment (or co-therapy) may be effected simultaneously, separately, or over a period of time (especially simultaneously).

“Simultaneously”, when referring to an administration type, means in the present application that the administration type concerned consists in the administration of two or more active ingredients and/or treatments at approximately the same time; wherein it is understood that a simultaneous administration will lead to exposure of the subject to the two or more active ingredients and/or treatments at the same time. When administered simultaneously, said two or more active ingredients may be administered in a fixed dose combination, or in an equivalent non-fixed dose combination (e.g. by using two or more different pharmaceutical compositions to be administered by the same route of administration at approximately the same time), or by a non-fixed dose combination using two or more different routes of administration; wherein said administration leads to essentially simultaneous exposure of the subject to the two or more active ingredients and/or treatments. When used in combination with standard background therapy, the aprocitentan would preferably be used “simultaneously”. “Fixed dose combination”, when referring to an administration type, means in the present application that the administration type concerned consists in the administration of one single pharmaceutical composition comprising the two or more active ingredients.

“Separately”, when referring to an administration type, means in the present application that the administration type concerned consists in the administration of two or more active ingredients and/or treatments at different points in time; wherein it is understood that a separate administration will lead to a treatment phase (e.g. at least 1 hour, notably at least 6 hours, especially at least 12 hours) where the subject is exposed to the two or more active ingredients and/or treatments at the same time; but a separate administration may also lead to a treatment phase where for a certain period of time (e.g. at least 12 hours, especially at least one day) the subject is exposed to only one of the two or more active ingredients and/or treatments. Separate administration especially refers to situations wherein at least one of the active ingredients and/or treatments is given with a periodicity substantially different from daily (such as once or twice daily) administration (e.g. wherein one active ingredient and/or treatment is given e.g. once or twice a day, and another is given e.g. every other day, or once a week or at even longer distances).

By administration “over a period of time” is meant in the present application the subsequent administration of two or more active ingredients and/or treatments at different times. The term in particular refers to an administration method according to which the entire administration of one of the active ingredients and/or treatments is completed before the administration of the other/the others begins. In this way it is possible to administer one of the active ingredients and/or treatments for several months before administering the other active ingredient(s) and/or treatment(s).

Aprocitentan can be used as medicament according to this invention, e.g. in the form of pharmaceutical compositions especially for enteral, or for parenteral administration.

Dosage forms suitable for enteral administration may be tablets or capsules (especially tablets) comprising a pharmaceutical composition comprising an efficacious amount of aprocitentan, or a pharmaceutically aceptable salt thereof.

For avoidance of doubt, for the present invention any amount/unit dose of aprocitentan refers to the amount/unit dose suitable for the administration of aprocitentan in free base form in such amount/unit dose. Such amount/unit dose may need to be adjusted in a pharmaceutical composition in case aprocitentan is present in such composition in a form different from anhydrous free base, such as a in form of a pharmaceutically acceptable salt; and/or a solvate such as a hydrate.

Whenever a certain dosage refers to a unit dose of a certain amount in mg, it is understood that such unit dose refers to such amount in mg of aprocitentan active ingredient in free base form. In case the active ingredient is administered e.g. in form of a pharmaceutically acceptable salt, it is understood that the respective amount of active pharmaceutical ingredient (e.g. said pharmaceutically acceptable salt) in a pharmaceutical composition will be adapted accordingly.

For avoidance of any doubt, it is understood that any pharmaceutical composition comprising aprocitentan in a pharmaceutically effective amount may additionally comprise further conventional excipients and/or additives, which may be used alone or in combination (quantum satis, i.e. wherein the maximum amounts of said further conventional ingredients and/or additives may need to be reduced to make up the total ww % of 100). It is understood that the total amount expressed in “ww %” of a certain composition is 100.

Reference is made to the extensive literature on the subject for these and other pharmaceutically acceptable excipients and procedures mentioned herein, see for example R. C. Rowe, P. J. Seskey, S. C. Owen, Handbook of Pharmaceutical Excipients, 5th edition, Pharmaceutical Press 2006; Remington, The Science and Practice of Pharmacy, 21st Edition (2005), Part 5, “Pharmaceutical Manufacturing” [published by Lippincott Williams & Wilkins].

The expression “ww %” (or % (w/w)) refers to a percentage by weight compared to the total weight of the composition considered. If not explicitly stated otherwise, the considered total weight is the total weight of the pharmaceutical composition.

The expression (wt/wt) relating to a ratio refers to a ratio by weight of the respective components.

In case a certain value is given as % value, in absence of further specification such value refers to ww %, or if in the context of purity, area % as measured by HPLC.

If not explicitly stated otherwise, any reference to aprocitentan refers to aprocitentan or a pharmaceutically acceptable salt form of aprocitentan, preferably to aprocitentan in free base form.

The term “pharmaceutical composition” is interchangeable with the terms “formulation”, or “composition”.

The term “treat” or “treatment” or “treating” used with reference to a disease means either that said disease is cured in the patient or animal; or that, although the animal or patient remains affected by the disease, part or all of the symptoms of said disease are either reduced or eliminated.

The terms “subject”, and likewise, “patient” refers to mammals, especially humans. Notably, the term “subject” refers to a human patient.

For avoidance of any doubt, any method of treatment of a certain disease or disorder such as RHT, said method comprising administering aprocitentan, or a pharmaceutically acceptable salt thereof, as set out in any one of embodiments 1) to 24) herein also discloses
aprocitentan, or a pharmaceutically acceptable salt thereof, for use in the treatment of said disease or disorder as set out in any one of embodiments 1) to 24) herein,
the use of aprocitentan, or of a pharmaceutically acceptable salt thereof, in the preparation of a medicament for the treatment of said disease or disorder as set out in any one of embodiments 1) to 24) herein;
   aprocitentan, or a pharmaceutically acceptable salt thereof, for use in such method of treatment of said disease or disorder as set out in any one of embodiments 1) to 24) herein;
   a medicament for the treatment of said disease or disorder as set out in any one of embodiments 1) to 24) herein, said medicament comprisong aprocitentan, or a pharmaceutically acceptable salt thereof; and the like.

Likewise, if aprocitentan, or a pharmaceutically acceptable salt thereof, is described as useful for the treatment of a certain disease or disorder such as RHT as set out herein, aprocitentan, or a pharmaceutically acceptable salt thereof, is likewise suitable:
for use in the preparation of a medicament for the treatment of said disease or disorder as set out herein;
for use in a method to treat of said disease or disorder, said method comprising administering aprocitentan, or a pharmaceutically acceptable salt thereof, as set out herein;
as a medicament for the treatment of said disease or disorder as set out herein, said medicament comprising aprocitentan, or a pharmaceutically acceptable salt thereof; and the like.

Particular embodiments of the invention are described in the following Examples, which serve to illustrate the invention in more detail without limiting its scope in any way.

### ABBREVIATIONS

ABP Ambulatory blood pressure
ABPM Ambulatory blood pressure monitoring
ACE Angiotensin-converting-enzyme as in ACE inhibitor (ACEI)
ARB(s) Angiotensin receptor blocker(s)
BP Blood pressure
BMI Body mass index
CCB(s) Calcium channels blocker(s)
DB(-WD)
Double blind (-withdrawal)
Diastolic blood pressure

DBP

ET Endothelin

RHT or rHT resistant hypertension

SB Single blind

SBP Systolic blood pressure

SBT Standardized background therapy

SiDBP Sitting diastolic blood pressure

SiSBP Sitting systolic blood pressure

UACR Urine albumin-creatinine ratio
uAOBP Unattended automated office blood pressure
uAOBPM Unattended automated office blood pressure measurement

### Example 1:

A method of manufacturing aprocitentan is described for example in WO2018/154101. Aprocitentan was used in the following clinical trial example in form of the stable crystalline form A disclosed therein.

### Example A): A Research Study to Show the Effect of Aprocitentan in the Treatment of Difficult to Control (Resistant) High Blood Pressure (Hypertension) and Find Out More About Its Safety (PRECISION).

A single, randomized study with three sequential treatment parts was designed. The study design addressed both the 4-week placebo-controlled efficacy of 12.5 and 25 mg aprocitentan (Part 1) and the durability of its effects in long-term active treatment with 25 mg aprocitentan for a further 32 weeks (Part 2) followed by a 12-week placebo-controlled withdrawal period with patients re-randomized to 25 mg or placebo (Part 3) (see for example Danaietash P et al; Identifying and treating resistant hypertension in PRECISION—a randomized long-term clinical trial with Aprocitentan. J Clin Hypertension 2022 (in press). Patients with uncontrolled BP (measured as unattended automated office BP) despite the use of three or more antihypertensive medications for at least 1 year were screened. They were switched to a single-tablet triple fixed combination antihypertensive therapy for at least 4 weeks before entering a single-blind placebo run-in period. The 4-week placebo run-in period further excluded placebo responders. The randomization period consisted of 3 sequential parts:
1) a 4-week double-blind part with aprocitentan 12.5 mg, 25 mg, or placebo (1:1:1 ratio);
2) a 32-week single-blind part with aprocitentan 25 mg; and
3) a 12-week randomized withdrawal part with aprocitentan 25 mg or placebo (1:1 ratio).

The purpose was to demonstrate the BP lowering effect of aprocitentan in RHT (Part 1) and the persistence of this effect (Parts 2 and 3) in chronic treatment.

The standardized byckground treatment (SBT) is a single-tablet triple fixed combination of a CCB (amlodipine), an ARB (valsartan), and a diuretic (hydrochlorothiazide); two dose strengths are available: 5/160/25 mg and 10/160/25 mg, respectively. The maximum tolerated dose strength is selected at the investigator's discretion during the treatment period of the study and must be kept stable for at least 1 week prior to randomization continuing through the end of the DB part and again during the DB-WD part. Patients who are treated with a β-blocker at screening will continue their treatment throughout the study.

Study treatment and SBT are to be taken every morning except on the morning of study visit days, where treatment is administered after the completion of the visit assessments and the measurement of BP.

Study treatment and SBT compliance are assessed throughout the study based on tablet counting. In addition, intake of SBT is monitored by assessing both participant urine via liquid chromatography with tandem mass spectrometric to detect valsartan, and by direct observed treatment intake, performed before start of the ambulatory BP monitoring (ABPM).

Trough uAOBP is measured at each visit with the same automated oscillometric sphygmomanometer (Microlife® WatchBP Office), which records 5 sitting BP readings (one per minute, first value excluded from the average), with the patient resting undisturbed, alone (unattended) in a quiet place for 5 minutes.

ABPM is performed over a 24-hour period with the Mobil-O-Graph NG device at baseline, and Weeks 4, 36, and 40. Systolic BP and diastolic BP are measured every 20 minutes from 06:00-23:00 and every 30 minutes from 23:00-06:00. Monitoring is initiated between 06:00 and 11:00.

### Study Treatments

Part 1: Double-blind, randomized parallel-group and placebo-controlled and lasts for 4 weeks. Subjects received (1:1:1 ratio), 25 or 12.5 mg aprocitentan, or placebo.
Part 2: Single-blind and single-arm, lasts for 32 weeks. All subjects received aprocitentan 25 mg.
Part 3: Double-blind withdrawal. All subjects completing part 2 and entering part 3 are re-randomized (1:1 ratio) to either aprocitentan 25 mg or placebo.

### Main Inclusion Criteria:

### Screening Period:

(i) Signed and dated ICF prior to any study-mandated procedure.
(ii) Male and female subjects; 18 years (or year of country specific majority) or older.
(iii) Historical documentation in the subject's medical records on uncontrolled BP despite at least 3 background antihypertensive medications within 1 year before screening visit.
(iv) Treated with at least 3 antihypertensive therapies of different pharmacological classes for at least 4 weeks before the screening visit (Visit 1).
(v) Mean SiSBP≥140 mmHg measured by uAOBPM.
(vi) Documentation in the subject's medical records of diagnosis of RHT according to the site's medical practice:
   Exclusion of secondary causes of hypertension (e.g., serum aldosterone, plasma renin activity, duplex/doppler ultrasonography, computer tomography angiography assessments are performed to exclude the secondary causes of hypertension),
   Adherence to medication (e.g., how the adherence was checked and/or monitored) to eliminate apparent RHT.
(vii) Women of childbearing potential are eligible only if the following applies:
   Negative pregnancy test at screening and at baseline (i.e., before randomization).
   Agreement to undertake pregnancy tests during the study and up to 30 days after randomized study treatment discontinuation.
   Agreement to use methods of birth control from screening up to at least 30 days after randomized study treatment discontinuation.
(viii) Mean trough SiSBP≥140 mmHg measured by AOBPM at the switch from background antihypertensive medications (i.e., at least 3 medications from different pharmacological classes]) to the standardized background antihypertensive therapy.

### Run-In Period (RI):

Switched to the standardized background antihypertensive therapy at least 4 weeks before the first RI visit.
Mean trough SiSBP≥140 mmHg as measured by uAOBPM.

### Randomization Period:

Stable dose of the standardized background antihypertensive therapy for at least 1 week before the end of the RI period.
Mean trough SiSBP≥140 mmHg measured by uAOBPM.
Subjects demonstrating ≥80% compliance (pill counting) to study treatment (i.e., placebo) as well as ≥80% compliance (pill counting) to the standardized background antihypertensive therapy during the RI period.

### Main Exclusion Criteria:

Apparent/pseudo RHT due to white coat effect, medical inertia, poor therapeutic adherence, or secondary causes of hypertension (except sleep apnea).
Confirmed severe hypertension (grade 3) defined as SiSBP≥180 mmHg and/or SiDBP≥110 mmHg as measured by uAOBPM at two different timepoints.
Pregnant or lactating subjects.
Clinically significant unstable cardiac disease at screening or in the past in the opinion of the investigator (exclusion of participants with significant or potential unstable cardiac disease).
Severe renal insufficiency.
Any known factor, disease or clinically relevant medical or surgical conditions that, in the opinion of the investigator, might put the subject at risk, interfere with treatment compliance, study conduct or interpretation of the results.
Treatment with any medication which may affect BP and/or treatment with high dose of loop diuretics (i.e., furosemide greater than 80 mg/day, or equivalent dosage of other loop diuretics).

### Endpoints

The primary efficacy endpoint is the change from baseline to Week 4 of DB treatment in mean trough SiSBP measured as uAOBP, where baseline is defined as the last available measurement before the start of DB treatment.

The key secondary efficacy endpoint is the change from DB-WD baseline (Week 36 or last available measurement before Week 36) to Week 40 in mean trough SiSBP measured as uAOBP.

Other secondary efficacy endpoints include
changes from baseline to Week 4 and from DB-WD baseline to Week 40 in mean trough sitting diastolic blood pressure (SiDBP) measured as uAOBP.
and changes in systolic/diastolic BP measured by ABPM, including changes from baseline to Week 4 of DB treatment in 24-h mean SBP and DBP, changes from baseline to Week 4 in daytime and night-time mean SBP and DBP, changes from DB-WD baseline (Week 36) to Week 40 in 24-h mean SBP and DBP, and changes from DB-WD baseline (Week 36) to Week 40 in daytime and night-time mean SBP and DBP.

Safety assessments include (treatment emergent) adverse events (TEAEs) including TEAEs leading to premature discontinuation of study treatment; adverse events of special interest (AESIs): hepatic disorders, anemia (or hemodilution), edema/fluid retention, and decompensation/aggravation of heart failure; treatment-emergent major adverse cardiovascular events (MACEs)/MACE-plus defined as cardiovascular death, non-fatal myocardial infarction (MI), or non-fatal stroke, or hear filure leading to hospitalization confirmed by the Central Adjudication Committee (CAC); as well as vital signs, body weight, clinical laboratory assessments, and 12-lead electrocardiograms.

### Statistical Analyses

In this design report, continuous endpoints are summarized by descriptive statistics and categorical variables by numbers and percentages. Categorical variables were compared using the Cochran-Mantel-Haenszel test for ordered variables and the Chi-square test for nominal variables. In addition, a stepwise multivariable logistic regression was performed for the patients with pseudo-RHT and those who were randomised to identify predictors of pseudo-RHT among the patient characteristics at screening (p<0.05 for inclusion in the model).

In the final analysis, three null hypotheses will be tested (See Figure S1 in the Data Supplement). The first two null hypotheses, which stipulate there is no difference between aprocitentan and placebo in the DB part, in the mean change from baseline to Week 4 in mean trough SiSBP measured as uAOBP (H10 for aprocitentan 25 mg and H20 for aprocitentan 12.5 mg).

### Results:

In the study, it was found that aprocitentan reduces blood pressure compared to placebo after 4 weeks of treatment, the effect is maintained and confirmed over a period of 48 weeks, and is generally well tolerated with no major safety concerns. Primary and key secondary endpoints were met with statistical significance and clinically meaningful results-the effect was consistent across multiple endpoints and methodologies of blood pressure monitoring.

To confirm a diagnosis of resistant hypertension and exclude pseudo resistant hypertension 1'965 patients were screened. During the 12-week screening period, qualifying patients were transitioned to standardized background antihypertensive therapy of a fixed-dose combination of a calcium channel blocker (amlodipine), an angiotensin receptor blocker (valsartan) and a diuretic (hydrochlorothiazide) for at least 4 weeks before entering a 4-week single-blind run-in period. In this period, placebo was added to the background antihypertensive therapy. Patients with systolic blood pressure consistently above 140 mmHg were then randomized to the first treatment part. Out of 1965 screened patients, 730 were randomized resulting in an overall inclusion failure rate of 63%. The most common reason for exclusion (44.4% of all screened patients) was failure to meet the BP inclusion criteria. These results underline the high proportion of pseudo-resistant hypertension among patients referred for RHT. Randomized patients had a medical history of comorbidities such as diabetes mellitus (about 53%), ischemic heart disease (about 30%), stroke (about 23%), congestive heart failure (about 19%), and/or sleep apnoea syndrome (about 14%). At screening, the later randomized patients received 3 (about 37%), 4 (about 46%) or equal or more than 5 (about 17%) main anti-hypertensive therapies. Said anti-hypertensive therapies comprised ACEIs or ARBs (total of about 98%: ACEIs about 37% and ARBs about 61%); diuretics (total about 86%, thereof about 11% mineralocorticoid receptor antagonists (MRA)); CCBs (about 83%); and beta blockers (about 61%).

In Part 1, the first double-blind treatment period of 4 weeks, a total of 730 patients were randomized to receive a tablet of aprocitentan 12.5 mg (N=243), 25 mg (N=243), or placebo (N=244) once daily. After 4 weeks of treatment, a statistically significant and clinically meaningful reduction in the primary endpoint measure of systolic blood pressure-measured at trough with an unattended automated office blood pressure (uAOBP) device-was observed in both the 12.5 mg (p<0.005) and 25 mg (p<0.005) aprocitentan groups compared to placebo.

Following the 4-week double-blind, placebo-controlled treatment period, patients entered Part 2, a single-blind treatment period, where all patients were treated with 25 mg aprocitentan for a further 32 weeks. The mean reduction from baseline in systolic blood pressure was maintained during this treatment period, for those patients who were on aprocitentan during Part 1. Patients switching from placebo to aprocitentan rapidly achieved the same blood pressure reduction as seen in Part 1.

This was followed by Part 3, a double-blind, placebo-controlled, randomized withdrawal treatment period where 614 patients were re-randomized to aprocitentan 25 mg or placebo for 12 weeks. After 4 weeks in the withdrawal period, the key secondary endpoint measure of systolic blood pressure increased significantly on placebo compared to aprocitentan 25 mg (p<0.0001). This provided replication of the treatment effect of aprocitentan and confirmed its durable antihypertensive effect.

The reduction in systolic and diastolic blood pressure assessed by measurement of unattended automated office blood pressure during the study, was confirmed by the 24-hour ambulatory blood pressure monitoring (ABPM), demonstrating BP reduction across the entire 24 h period (notably during the night).

Aprocitentan was generally well tolerated with no major safety concerns in this patient population at both doses and with a low discontinuation from study treatment due to an adverse event in the first 4 weeks double-blind study period: 2.5% and 2.0% for aprocitentan 12.5 mg and 25 mg groups respectively versus 0.8% in the placebo group. Treatment-emergent adverse events (TEAEs) during the 4-week double-blind study period were reported in 27.6% and 36.7% of the patients treated with 12.5 and 25 mg aprocitentan, respectively, versus 19.4% in the placebo group. The most frequent TEAEs reported over 3% incidence and higher than placebo was edema/fluid retention. There were no additional emerging safety findings in the subsequent treatment period taking the total to 48 weeks. Importantly, the overall incidence of Major Adverse Cardiac Events (MACE) reflected the expected occurrence in this patient population. Approximately 30% of patients developed edema/fluid retention, at one time point during the entire study duration, with >95% being mild to moderate in intensity. Two (<1%) of these adverse events were serious (both on aprocitentan 25 mg). Only seven (<1%) of patients discontinued treatment due to edema/fluid retention. Edema/fluid retention was mostly reported by patients within the first 4-week double-blind study period (9.1% and 18.4% for aprocitentan 12.5 mg and 25 mg groups respectively, versus 2.1% in the placebo group).

Results of the PRECISION trial have been published in more detail by Schlaich et al., Lancet 2022; 400:1927-37 and associated supplementary material (both documents are herewith incorporated by reference), as follows: Patient characteristics were similar across all treatment groups at randomisation and re-randomisation. They were representative of the patients affected by resistant hypertension regarding age and comorbidities. Black or African American patients represented 11% of all randomly assigned participants and 37% (78 of 211) of the participants from the USA, which is higher than the distribution of Black people in the USA. At screening, 63% of all patients who were randomly assigned were prescribed four or more antihypertensive drugs and 63% were receiving a ß blocker, as described previously. At randomisation, 516 (71%) of 730 patients received the highest dose of standardised background therapy (amlodipine 10 mg) and 423 (58%) of 730 patients continued their β blocker treatment.

The time course of changes in office SBP and DBP during the 3 study parts illustrates the short-term (4 weeks) and sustained (up to 48 weeks) blood pressure lowering effects of aprocitentan. After 4 weeks of treatment in the double-blind part 1, the primary endpoint was met. The least square mean (SE) change in office SBP at 4 weeks was −15.3 (0.9) mm Hg for aprocitentan 12.5 mg, −15.2 (0.9) mm Hg for aprocitentan 25 mg, and −11.5 (0.9) mm Hg for placebo, for a difference versus placebo of −3.8 (1.3) mm Hg (97.5% CI −6.8 to −0.8, p=0.0042) and −3.7 (1.3) mm Hg (−6.7 to −0.8, p=0.0046), respectively. Office DBP also decreased with both aprocitentan doses compared with placebo (−3.9 mm Hg, 95% CI −5.6 to −2.3 for the 12.5 mg dose; −4.5 mm Hg, 95% CI −6.1 to −2.9 for the 25 mg dose, respectively). Office SBP and DBP were maintained during part 2 in patients previously receiving aprocitentan and decreased within the first 2 weeks of part 2 before stabilising in those previously receiving placebo. In part 3, office SBP after 4 weeks of withdrawal (the key secondary endpoint) increased significantly with placebo compared with aprocitentan (5.8 mm Hg, 95% CI 3.7 to 7.9, p<0.0001). Office DBP also increased with placebo compared with aprocitentan (5.2 mm Hg, 95% CI 3.8 to 6.6, p<0.0001). The difference between the two groups remained up to week 48.

The results of all sensitivity and supportive analyses confirmed the robustness of the main analysis. In particular, the exclusion of patients resulting from the premature discontinuation of double-blind treatment or the addition or increase in dose of a diuretic or the use of rescue medication for blood pressure increase did not affect the primary and key secondary results.

The results from ambulatory blood pressure monitoring confirmed those derived from office measurements. At the end of part 1, aprocitentan, after placebo correction, decreased both the 24 h ambulatory SBP (−4.2 mm Hg, 95% CI −6.2 to −2.1 for the 12.5 mg dose; −5.9 mm Hg, −7.9 to −3.8 for the 25 mg dose) and DBP (−4.3 mm Hg, 95% CI −5.7 to −3.0 for the 12.5 mg dose; −5.8 mm Hg, 95% CI −7.1 to-4.5 for the 25 mg dose). The placebo-corrected SBP lowering effect was −5.1 mm Hg and −7.4 mm Hg during the night time and −3.8 mm Hg and −5.3 mm Hg during the daytime, for the 12.5 mg and 25 mg doses, respectively.

In part 3, after 4 weeks of withdrawal (week 40), both the 24 h ambulatory SBP and DBP increased with placebo compared with aprocitentan (6.5 mm Hg, 95% CI 4.6 to 8.5; 6.8 mm Hg, 95% CI 5.5 to 8.0, respectively).

For the primary and key secondary efficacy analyses, a treatment effect consistent with that in the overall study population was observed across the majority of subgroups. Notably, a greater decrease in SBP was seen at week 4 for older patients (aged >75 years), and for patients with macro-albuminuria (urine albumin-creatinine ratio (UACR) >300 mg/g) and chronic kidney disease stage 3-4 (estimated glomerular filtration rate [eGFR] 15 to <60 mL/min per 1.73 m2). No difference in treatment effect was detected between patients with or without β-blocker treatment at screening. Regarding race, Black or African American patients compared with other patients tended to have a lower response to aprocitentan at week 4, and a stronger response at week 40, after 4 weeks of placebo-controlled withdrawal. The ambulatory blood pressure monitoring results in Black or African American patients were consistent with those observed in the overall population across study parts.

At the end of part 1, a reduction of −28% and −31% in the urine albumin-creatinine ratio (UACR) was observed for the 12.5 mg and 25 mg aprocitentan groups, respectively, and an increase of 5% was observed for the placebo group. In part 2, the reduction was maintained for all patients. In part 3, after 4 weeks of withdrawal, the ratio increased with placebo compared with aprocitentan. This antiproteinuric effect of aprocitentan tended to be greater in patients with chronic kidney disease stage 3-4 versus patients with an eGFR of 60 mL/min per 1.73 m² or more.

Aprocitentan was well tolerated. The most frequent adverse event was oedema or fluid retention occurring mainly during the first 4 weeks of treatment. Before randomisation, 70 (10%) of 730 patients had an ongoing medical condition of oedema or fluid retention and 35 (5%) of 730 had experienced an adverse event of oedema or fluid retention. Oedema or fluid retention was reported more frequently with aprocitentan than with placebo in a dose-dependent manner (9.1%, 18.4%, and 2.1% for patients receiving aprocitentan 12.5 mg, 25 mg, and placebo, during the 4-week part 1, respectively; 18.2% for patients receiving aprocitentan 25 mg during the 32-week part 2; and 2.6% and 1.3% for patients on aprocitentan 25 mg and placebo, during the 12-week part 3, respectively). Oedema or fluid retention was generally mild to moderate and diuretic treatment was added as needed. Oedema or fluid retention was more frequent in patients with chronic kidney disease stage 3-4.

Discontinuation due to oedema or fluid retention was reported for seven patients receiving aprocitentan 25 mg during parts 1-3 (one of 245 patients in part 1, five of 704 patients in part 2, and one of 310 patients in part 3.

A total of 13 deaths were reported, two of which were not considered treatment-emergent. Of the 11 treatment-emergent deaths, none were regarded by the investigators to be related to study treatment; five were cardiovascular deaths, five were COVID-19-related, and one patient died of procedural intestinal perforation.

Eleven patients required admission to hospital for heart failure (two [0.8%] of 245 receiving aprocitentan 25 mg during part 1; six [0.9%] of 704 during part 2; and two [0.6%] of 310 receiving aprocitentan 25 mg and one [0.3%] of 303 receiving placebo during part 3); none of the cases were fatal. All patients had a high-risk cardiovascular medical history including diabetes (11 [100%] of 11), chronic kidney disease stage 3-4 (6 [55%] of 11), and pre-existing heart failure (5 [45%] of 11). Two (18%) of 11 patients discontinued from study treatment due to heart failure. Major cardiovascular events included the five cardiovascular deaths.

No signs of hepatotoxicity were observed. Haemoglobin concentrations decreased and estimated plasma volume increased to a similar degree with both aprocitentan doses (−8.0 g/L,-8.5 g/L, and −0.4 g/L for haemoglobin; and 10.5%, 11.2%, and 0.51% for estimated plasma volume, with aprocitentan 12.5 mg, aprocitentan 25 mg, and placebo, respectively) during part 1, stabilised during part 2 and reversed upon withdrawal during part 3. Slight increases in N-terminal pro-brain natriuretic peptide (NT-proBNP) and mid-regional pro-atrial natriuretic peptide (MR-proANP) were observed in part 1 with aprocitentan, followed by stabilisation during part 2 and reversal during part 3. A moderate increase in bodyweight with both aprocitentan doses and a decrease with placebo were observed during part 1. The eGFR decreased slightly with aprocitentan in part 1 versus placebo, stabilised in part 2, and decreased further in the aprocitentan group in part 3 while remaining stable in the placebo group. A marginal decrease in heart rate was apparent in all treatment groups during part 1 and maintained during part 2.

Schlaich et al. further discuss that a substantial reduction in urine albumin-creatinine ratio of 28% and 31% was observed for the 12.5 mg and 25 mg aprocitentan doses, respectively, whereas urine albumin-creatinine ratio increased by 5% with placebo in the double-blind part 1. This antiproteinuric effect of aprocitentan tended to be greater in patients with chronic kidney disease stage 3-4 versus patients with eGFR of 60 mL/min per 1.73 m² or higher (−46% versus −26%), perhaps highlighting the potential of aprocitentan to reduce organ damage even in patients with moderate-to-severe chronic kidney disease, in whom blood pressure control is particularly difficult to achieve. Importantly, the antiproteinuric effect was sustained during the single-blind part 2, during which all participants were on 25 mg aprocitentan.

### Further Data of the PRECISION Trial:

Between-treatment analyses for changes from baselines in 24 h, daytime and night time SBP/DBP at Week 4 and Week 40 show meaningful and consistent SBP/DBP reductions for aprocitentan groups compared to placebo. Of particular clinical relevance is the difference to placebo in night-time mean SBP (-6.6 mmHg) observed at Week 4 with aprocitentan 25 mg.

An antiproteinuric effect of aprocitentan (i.e., reductions in UACR: −30% and −34% in the aprocitentan 12.5 mg and 25 mg groups, respectively) was demonstrated in this study, as early as Week 4 and maintained over 48 weeks of treatment, while no change was observed with placebo. This effect was more pronounced in subjects with CKD stage 3-4 (−26% with aprocitentan 12.5 mg and −45% with 25 mg); it was also more pronounced in subjects with baseline microalbuminuria (−43% with 12.5 mg and −45% with 25 mg). The greatest effect (−48% with 12.5 mg and −61% with 25 mg) was observed in subjects with macroalbuminuria.

When all available data in the overall population and in subgroups of particular interest are considered, an exposure-and dose-response relationship between 12.5 mg and 25 mg is observed for all Week 4 secondary BP efficacy endpoints:
in the overall population, aprocitentan 25 mg achieved numerically greater reduction (than 12.5 mg) in all BP endpoints except for the primary endpoint;
the benefit of 25 mg versus 12.5 mg is particularly evident in night-time BP (collected via ABPM), which is a well-known major predictor of cardiovascular outcome;
the difference between 25 mg and 12.5 mg is particularly patent in the CKD stage 3-4 and Black or African American patient subgroups, which are in high medical need when their hypertension becomes difficult to control.

### Dose-Response in Subpopulations:

In two subpopulation groups: (i) CKD stage 3-4 and (ii) Black or African American, a consistent dose-response was observed for all endpoints listed above, including the percent increase in BP reduction for the 25 mg dose compared to 12.5 mg.

In Black or African Americans, least square (LS) mean reductions from baseline to Week 4 in BP were consistently greater with aprocitentan 25 mg than with aprocitentan 12.5 mg for all endpoints. An additional gain (64% to 145%) of the 25 mg over 12.5 mg with ABPM was observed in this subpopulation, with differences up to −5.90 mmHg (i.e., >100% further decrease in BP with 25 mg compared to 12.5 mg in night-time SBP). In subjects with CKD stage 3-4, least square (LS) mean reductions from baseline to Week 4 in BP were consistently greater with aprocitentan 25 mg than with aprocitentan 12.5 mg for all endpoints, with differences up to −5.88 mmHg for primary endpoint SiSBP (represents a 44% further decrease compared with 12.5 mg).

### Dose-Response Relationship in UACR:

In a population with difficult-to control hypertension (mean age of 62 years, obesity [69.3% of subjects], >50% with diabetes mellitus, 22% with CKD stage 3-4), evaluation of UACR is important. The PRECISION trial did not implement any inclusion criterion for UACR. The geometric mean UACR at baseline was 26.6 mg/g in the overall population. At Week 4, placebo-corrected reductions in UACR of 30% and 34% with aprocitentan 12.5 mg and 25 mg were noted, respectively. However, in the subgroup of subjects with macroproteinuria (>300 mg/g UACR; 12.6% of subjects), a clear dose-response relationship was observed with a placebo-corrected reduction of 48% with 12.5 mg vs 61% with 25 mg.

1. A method of treating hypertension in a human subject in need thereof, wherein the method comprises administering to the human subject a pharmaceutical composition comprising a clinically proven effective amount of aprocitentan, or a pharmaceutically acceptable salt thereof.

2. The method according to claim 1, wherein said human subject in need thereof is a human subject receiving standard background therapy for treatment of hypertension, wherein said background therapy comprises angiotensin receptor blocker (ARB) or an ACE inhibitor.

3. The method according to claim 1, wherein aprocitentan is administered in combination with at least three other antihypertensive drugs independently selected from the group consisting of:
an angiotensin receptor blocker, or a pharmaceutically acceptable salt thereof;
an ACE inhibitor, or a pharmaceutically acceptable salt thereof;
a calcium channel blocker, or a pharmaceutically acceptable salt thereof;
a diuretic; and
a beta blocker.

4. The method according to claim 1, wherein administering said clinically proven effective amount of aprocitentan results in a reduction of systolic blood pressure of at least about 15 mmHg from baseline after 4 weeks of treatment, wherein said reduction is measured by unattended Automated Office Blood Pressure Measurement (uAOBPM) at trough.

5. The method according to claim 1, wherein the administering said clinically proven effective amount of aprocitentan for an additional 32 weeks after said initial 4 weeks of treatment results at least in the maintenance of the mean reduction from baseline in systolic blood pressure for such additional 32 weeks.

6. The method according to claim 4, wherein the clinical effect on systolic blood pressure is confirmed after 4 weeks of a withdrawal period, wherein the mean systolic blood pressure increases in human subjects administered with placebo compared to the mean systolic blood pressure in human subjects who continue to be administered with aprocitentan at a dose of 25 mg per day; wherein said increase is at least about 6 mmHg; wherein said increase is measured by unattended Automated Office Blood Pressure Measurement (uAOBPM) at trough.

7. The method according to claim 4, wherein said clinical effect on blood pressure reduction is confirmed by a mean reduction from baseline blood pressure after 4 weeks of treatment, wherein said reduction is measured by 24 hours ambulatory blood pressure monitoring (24 h ABPM); wherein
systolic blood pressure is reduced by at least about 6 mmHg, and/or
diastolic blood pressure is reduced by at least about 6 mmHg,
wherein said clinically proven effective amount of aprocitentan is 12.5 mg per day; or
systolic blood pressure is reduced by at least about 8 mmHg, and/or
diastolic blood pressure is reduced by at least about 7 mmHg,
wherein said clinically proven effective amount of aprocitentan is 25 mg per day.

8. The method according to claim 4, wherein said clinical effect on blood pressure reduction is confirmed by a mean reduction from baseline blood pressure after 4 weeks of treatment, wherein said reduction is measured by nighttime ambulatory blood pressure monitoring (nighttime ABPM); wherein
systolic blood pressure is reduced by at least about 6 mmHg, and/or
diastolic blood pressure is reduced by at least about 6 mmHg,
wherein said clinically proven effective amount of aprocitentan is 12.5 mg per day; or
systolic blood pressure is reduced by at least about 8 mmHg, and/or
diastolic blood pressure is reduced by at least about 7 mmHg,
wherein said clinically proven effective amount of aprocitentan is 25 mg per day.

9. The method according to claim 1, wherein the administering said clinically proven effective amount of aprocitentan results in a mean placebo corrected reduction of blood pressure after 4 weeks of treatment, wherein said reduction is measured by 24 hours ambulatory blood pressure monitoring (24h ABPM); wherein.
systolic blood pressure is reduced by at least about 4 mmHg, and/or
diastolic blood pressure is reduced by at least about 5.5 mmHg,
wherein said clinically proven effective amount of aprocitentan is 12.5 mg per day; or.
systolic blood pressure is reduced by at least about 4 mmHg, and/or
diastolic blood pressure is reduced by at least about 5.5 mmHg,
wherein said clinically proven effective amount of aprocitentan is 25 mg per day.

10. The method according to any one of claims 1 to 8 claim 1, wherein the administering said clinically proven effective amount of aprocitentan results in a mean placebo corrected reduction of blood pressure after 4 weeks of treatment, wherein said reduction is measured by nighttime ambulatory blood pressure monitoring (nighttime ABPM); wherein
systolic blood pressure is reduced by at least about 4 mmHg, and/or
diastolic blood pressure is reduced by at least about 4 mmHg,
wherein said clinically proven effective amount of aprocitentan is 12.5 mg per day;
systolic blood pressure is reduced by at least about 4 mmHg, and/or
diastolic blood pressure is reduced by at least about 5.5 mmHg,
wherein said clinically proven effective amount of aprocitentan is 25 mg per day.

11. The method according to claim 4, wherein the reduction of blood pressure is confirmed after 4 weeks of a withdrawal period, wherein the mean blood pressure increases in human subjects administered with placebo compared to the respective mean blood pressure in human subjects who continue to be administered with aprocitentan at a dose of 25 mg per day; wherein said mean blood pressure increase in human subjects administered with placebo is measured by nighttime ambulatory blood pressure monitoring (nighttime ABPM); wherein.
systolic blood pressure is increased by at least about 8.5 mmHg, and/or
diastolic blood pressure is increased by at least about 7.5 mmHg.

12. The method according to claim 1, wherein said method of treating hypertension in a human subject in need thereof comprises administering to the human subject a pharmaceutical composition comprising a clinically proven safe and clinically proven effective amount of aprocitentan.

13. The method according to claim 1, wherein the clinically proven effective amount of aprocitentan is 12.5 mg or 25 mg per day of aprocitentan.

14. The method according to claim 1, wherein the clinically proven effective amount of aprocitentan is 12.5 mg per day of aprocitentan.

15. The method according to claim 1, wherein the clinically proven effective amount of aprocitentan is 25 mg per day of aprocitentan.

16. The method according to claim 1, wherein said hypertension is difficult to control hypertension or resistant hypertension.

17. The method according to claim 2, wherein said human subject in need thereof is a human subject receiving standard background therapy for treatment of hypertension wherein said background therapy further comprises a calcium channel blocker (CCB) and/or a diuretic.

18. The method according to claim 17, wherein said human subject in need thereof is a human subject receiving standard background therapy for treatment of hypertension wherein said background therapy further comprises a beta blocker.

19. The method according to claim 3, wherein the diuretic is a loop diuretic, a potassium-sparing diuretic, a carbonic anhydrase inhibitor, or a thiazide-like diuretic.

20. The method according to claim 3, wherein the at least three other antihypertensive drugs are independently selected from the group consisting of:
an angiotensin receptor blocker selected from the group consisting of valsartan, losartan, candesartan, irbesartan, telmisartan, eprosartan, olmesartan, azilsartan, and fimasartan, or a pharmaceutically acceptable salt thereof;
an ACE inhibitor selected from the group consisting of enalapril, ramipril, quinapril, perindopril, lisinopril, benazepril, imidapril, trandolapril, and cilazapril, or a pharmaceutically acceptable salt thereof;
a calcium channel blocker selected from the group consisting of amlodipine, aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, and pranidipine, or a pharmaceutically acceptable salt thereof;
a diuretic selected from the group consisting of furosemide, bumetanide, ethacrynic acid, torsemide, spironolactone, eplerenone, finerenone, acetazolamide, methazolamide, chlorthalidone, hydrochlorothiazide, chlorothiazide, indapamide, and metolazone; and
a beta blocker selected from the group consisting of acebutolol, atenolol, bisoprolol, metoprolol, nadolol, nebivolol, and propranolol.

21. The method according to claim 20, wherein the diuretic is chlorthalidone or hydrochlorothiazide.

22. The method according to claim 4, wherein the clinically proven effective amount of aprocitentan is 12.5 mg per day of aprocitentan.

23. The method according to claim 12, wherein the clinically proven safe and clinically proven effective amount of aprocitentan is 12.5 mg per day of aprocitentan.

24. The method according to claim 1, wherein said hypertension is difficult to control hypertension.

25. The method according to claim 24, wherein said human subject in need thereof is a human subject receiving standard background therapy for treatment of hypertension wherein said background therapy comprises treatment with at least two antihypertensive agents of different classes, wherein one of said antihypertensive agents is a diuretic.

26. The method according to claim 24, wherein administering said clinically proven effective amount of aprocitentan results in a reduction of systolic blood pressure of at least about 15 mmHg from baseline after 4 weeks of treatment, wherein said reduction is measured by unattended Automated Office Blood Pressure Measurement (uAOBPM) at trough.

27. The method according to claim 24, wherein administering said clinically proven effective amount of aprocitentan results in a mean placebo corrected reduction of blood pressure after 4 weeks of treatment, wherein said reduction is measured by nighttime ambulatory blood pressure monitoring (nighttime ABPM); wherein
systolic blood pressure is reduced by at least about 4 mmHg, and/or
diastolic blood pressure is reduced by at least about 4 mmHg,
wherein said clinically proven effective amount of aprocitentan is 12.5 mg per day.

28. The method according to claim 24, wherein the clinically proven effective amount of aprocitentan is 12.5 mg per day of aprocitentan.

29. The method according to claim 26, wherein said method of treating difficult to control hypertension in said human subject in need thereof comprises administering to the human subject a pharmaceutical composition comprising a clinically proven safe and clinically proven effective amount of aprocitentan; wherein the clinically proven safe and clinically proven effective amount of aprocitentan is 12.5 mg per day of aprocitentan.
